(19) United States
(12) Patent Application Publication
(10) Pub. No.: US 2020/0407797 A1
(43) Pub. Date: Dec. 31, 2020

(54) COMPOSITIONS AND METHODS FOR MUTATIONS ASSOCIATED WITH SUDDEN UNEXPECTED DEATH IN PEDIATRICS

(71) Applicant: Children's Medical Center Corporation, Boston, MA (US)

(72) Inventors: Richard D. Goldstein, Boston, MA (US); Ingrid A. Holm, Boston, MA (US); Annapurna Poduri, Boston, MA (US); Catherine A. Brownstein, Boston, MA (US); Gerard T. Berry, Boston, MA (US)

(73) Assignee: Children's Medical Center Corporation, Boston, MA (US)

(21) Appl. No.: 16/979,084

(22) PCT Filed: Mar. 8, 2019
(86) PCT No.: PCT/US2019/021366

§ 371 Date: Sep. 8, 2020

Related U.S. Application Data
(60) Provisional application No. 62/641,166, filed on Mar. 9, 2018.

(51) Int. Cl. C12Q 1/6883

(52) U.S. Cl. CPC C12Q 1/6883; C12Q 2600/16

(57) ABSTRACT

The invention features panels of genes associated with Sudden Unexpected Death in Pediatrics (SUDP), and methods of using such panels to identify a cause of death, and to select children at risk of SUDP for therapies to treat pathologies that predispose them to SUDP.

### CROSS-REFERENCE TO RELATED APPLICATIONS

This application claims the benefit of and priority to the following U.S. Provisional Application No. 62/641,166, filed Mar. 9, 2018, the entire contents of which are incorporated herein by reference.

### BACKGROUND OF THE INVENTION

Sudden unexpected death in pediatrics (SUDP) is defined as the sudden, unexpected death of a child that remains unexplained after a thorough death scene investigation and autopsy, and includes sudden infant death syndrome (SIDS), sudden unexpected infant death (SUID), and sudden unexplained death in childhood (SUDC). SUDP is responsible for more deaths than cancer or heart disease in infants and children. SUDP is a diagnosis of exclusion, and investigations conventionally focus on evidence of asphyxia in sleep environments or practices, in which these deaths typically occur. Among children ultimately diagnosed with SUDP, those dying unexpectedly who are <3 years of age are the least likely to have explanations found with the current standard approach to investigation. Such unexplained deaths are the subject of police and medical examiner investigations, and are a lasting source of pain for parents, particularly where there are siblings who are at risk for SUDP.

### SUMMARY OF THE INVENTION

As described below, the present invention features panels of genes associated with Sudden Unexpected Death in Pediatrics (SUDP), and methods of using such panels to identify a cause of death, and to select children at risk of SUDP for therapies to treat pathologies that predispose them to SUDP.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include sudden unexplained death in pediatrics, sudden infant death syndrome (SIDS), sudden unexpected infant death (SUID), and sudden unexplained death in childhood (SUDC)

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in sequence, expression level or activity that is associated with a disease or disorder. Exemplary markers include genes present in the panels described herein, as well as the proteins they encode.

By “mutation” is meant a change in a polypeptide or polynucleotide sequence relative to a reference sequence. In some embodiments, the reference sequence is a wild-type sequence. Exemplary mutations include point mutations, missense mutations, amino acid substitutions, and frameshift mutations, as well as deletions or duplications, in all or part of a gene. A “loss-of-function mutation” is a mutation that decreases or abolishes an activity or function of a polypeptide. A “gain-of-function mutation” is a mutation that enhances or increases an activity or function of a polypeptide. In one embodiment, the mutation is a single nucleotide polymorphism (SNP).

“Primer set” means a set of oligonucleotides that may be used, for example, for amplifying a polynucleotide of interest (e.g., by PCR). A primer set would include, for example, at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a pie chart that describes various phenotypes of SUDP.

FIG. 2 shows hippocampal developmental lesions in two infants with SIDS and variants in SCN1A. FIG. 2A. Case 1: SIDS infant with SCN1A c.2045G>T, p.Gly682Val variant. Low power photograph of the hippocampus shows the abnormal dentate gyrus with a region of focal bilamination highlighted in the black rectangle. (Haematoxylin+Eosin stain, ×4). FIG. 2B. Case 1: Focal dentate bilamination with two layers of granule cells and intervening neuropil (arrow). Other abnormalities include mild hyperconvolution of the dentate gyrus, immature neuronal-like precursors in the subgranular zone, ectopic granule cells in the molecular layer and hilus, and mild hilar gliosis. (Haematoxylin+Eosin stain, ×20). FIG. 2C. Control dentate gyrus in an age-matched infant showing the normal single layer of dentate gyrus granule cells in row. (Haematoxylin+Eosin stain, ×20). FIG. 2D. Case 2: SIDS infant with c.3886T>A, Leu1296Met and c. 3924A>T, Glu1308Asp variants in cis: Low power photograph of the hippocampus shows dentate gyrus bilamination in two foci (black rectangle, arrow). The dentate gyrus is slightly hyperconvoluted. Other abnormalities include immature neuronal-like precursors in the subgranular zone, ectopic granule cells in the molecular layer and hilus, and mild hilar gliosis. (Haematoxylin+Eosin stain, ×2). FIG. 2E. Case 2: Focal dentate bilamination (and trilamination) (arrow) in the rectangle from FIG. 2D. (Haematoxylin+Eosin stain, ×20). FIG. 2F. Hilar gliosis in Case 2, demonstrated with standard immunocytochemistry for glial fibrillary acidic protein (GFAP) to label reactive astrocytes (short arrow). GFAP, ×40. Abbreviations: CP, choroid plexus; DG, dentate gyrus, ML, LGN, lateral geniculate nucleus; molecular layer.

FIG. 3 provides a model of SCN1A protein and predicted pathogenic variants seen in two cases of SIDS. Case 1: c.2045G>T, p.Gly682Val. Case 2: c.3886T>A, Leu1296Met and c. 3924A>T, Glu1308Asp (present in cis configuration). Pathologic variants in close proximity to G682V, affecting the same transmembrane domain, have been associated with Dravet syndrome (D674G)¹³ and borderline severe myoclonic epilepsy of infancy, (T685LfsX5)¹⁴ and are depicted by dots on the model to show relative position. Previously reported pathologic variants in close proximity to Leu1296 are W1284X and F1289del; the patients were diagnosed with severe myoclonic epilepsy of infancy.

FIG. 4 provides a functional evaluation of SCN1A variants. A. Representative whole-cell sodium currents recorded from tsA201 cells expressing either WT Na_{V}1.1 or SIDS associated variants. B. Current-voltage relationships of WT Na_{V}1.1 and SIDS associated variants. All data are expressed as mean±SEM for 14-15 measurements. Statistical differences were determined by ANOVA (*, p<0.05 for both variants compared to WT; p<0.05 between WT and L1296M/E1308D).

### DETAILED DESCRIPTION OF THE INVENTION

The invention features panels of genes associated with Sudden Unexpected Death in Pediatrics (SUDP) and methods of using such panels.

The invention is based, at least in part, on the discovery that SUDP has a genetic basis and is related to sequence alterations in genes associated with neurodevelopment, epilepsy, cardiac function, metabolism, infectious mechanisms, and other pathways important for survival. Whole exome sequencing (WES) was performed on samples derived from SUDP. This identified panels of genes associated with SUDP. Whole exome sequencing was carried out concurrently with extensive pathological phenotyping and obtaining detailed family and medical histories. Variants on the panel of genes were assessed for pathogenicity using standard guidelines, inheritance assessed through trio analysis, and CLIA laboratory confirmation. Reportable variants were identified in over 30% of SUDP cases. Exomes were also analyzed for novel rare variants in candidate genes without known disease association in categories related to known disease genes. These genes indicated the role of epilepsy, changes in the hippocampus, and ion channel genes in SUDP. Whole genome sequencing was also carried out to assess indels and non-coding regions in genes on the panel. Initial identification of single nucleotide variants (SNVs) and indels followed by evaluation of candidate variants for relevance to the phenotype.

SCN1A variants were identified in two infants who died of Sudden Infant Death Syndrome (SIDS) from an exome sequencing study of 10 cases of SIDS with hippocampal abnormalities, but no history of seizures. One harbored SCN1A G682V, and the other had two SCN1A variants in cis: L1296M and E1308D, a variant previously associated with epilepsy. Functional evaluation in a heterologous expression system demonstrated partial loss-of-function for both G692V and the compound variant L1296M/E1308D. These cases represent a novel association between SCN1A and SIDS, extending the SCN1A spectrum from epilepsy to SIDS. These findings also provide insights into SIDS and support genetic evaluation focused on epilepsy genes in SIDS.

### SUDP Panels

The invention provides panels of polynucleotides or polypeptides associated with SUDP. In one embodiment, genes of Table 1 or Table 2 are characterized in a biological sample of a subject affected by SUDP, such subjects include a pediatric proband who has died suddenly and unexpectedly where the cause of death is unknown, as well as first and second-degree relatives of that proband. Such first and second-degree relatives may have a mutation in a gene of Table 1 or Table 2 that predisposes them or their progeny to SUDP.

TABLE 1
SUDP Genes

| Gene | | | | |
|---|---|---|---|---|
| Symbol | Gene Name | Associated Condition | Inheritance | Category |
| ABCC9 | ATP-BINDING CASSETTE, | Atrial fibrillation, familial, | AD | Cardiac |
| | SUBFAMILY C, MEMBER 9 | 12; cardiomyopathy, dilated | | |
| | (SULFONYLUREA | | | |
| | RECEPTOR 2; SUR2) | | | |
| ACADM | ACYL-CoA | Acyl-CoA dehydrogenase, | AR | Metabolic |
| | DEHYDROGENASE, | medium chain, deficiency of | | |
| | MEDIUM-CHAIN | | | |
| ACADVL | ACYL-CoA | VLCAD deficiency | AR | Metabolic |
| | DEHYDROGENASE, VERY | | | |
| | LONG-CHAIN | | | |
| ACTA1 | ACTIN, ALPHA, | Myopathy, actin, | AD, AR | Cardiac |
| | SKELETAL MUSCLE 1 | congenital; Nemaline | | |
| | | myopathy 3, autosomal | | |
| | | dominant or recessive; | | |
| | | pathways are | | |
| | | Arrhythmogenic right | | |
| | | ventricular cardiomyopathy | | |
| | | (ARVC) | | |
| ACTC1 | ACTIN, ALPHA, CARDIAC | Cardiomyopathy, | AD | Cardiac |
| | MUSCLE | hypertrophic, 11; | | |
| | | Cardiomyopathy, dilated, | | |
| | | 1R; Left ventricular | | |
| | | noncompaction 4 | | |
| ACTN2 | ACTININ, ALPHA-2 | Cardiomyopathy, | AD | Cardiac |
| | | hypertrophic, 23; | | |
| | | Cardiomyopathy, dilated, | | |
| | | 1AA | | |
| AKAP9 | A-KINASE ANCHOR | ?Long QT syndrome-11 - | AD | Cardiac |
| | PROTEIN 9 | sudden death | | |
| ALDH7A1 | ALDEHYDE | Epilepsy, pyridoxine- | AR | Epilepsy |
| | DEHYDROGENASE 7 | dependent | | |
| | FAMILY, MEMBER A1 | | | |
| ALDOB | ALDOLASE B, FRUCTOSE- | Fructose intolerance, | AR | Metabolic |
| | BISPHOSPHATE | hereditary | | |
| ANK2 | ANKYRIN 2 | Long QT syndrome 4; | AD | Cardiac |
| | | Cardiac arrhythmia, | | |
| | | ankyrin-B-related | | |
| ANKRD1 | ANKYRIN REPEAT | Ankrd1-Related Dilated Cardiomyopathy | | Cardiac |
| | DOMAIN-CONTAINING | | | |
| | PROTEIN 1 | | | |
| ARIX | ARISTALESS HOMEOBOX, | Sudden Infant Death Syndrome (SIDS) | | SIDS |
| (PHOX2A) | DROSOPHILA, HOMOLOG | Susceptibility Pathways | | |
| | OF | | | |
| ARX | ARISTALESS-RELATED | Epileptic encephalopathy, | XL, XLR | Epilepsy |
| | HOMEOBOX, X-LINKED | early infantile, 1; | | |
| | | Hydranencephaly with | | |
| | | abnormal genitalia; | | |
| | | Lissencephaly, X-linked 2 | | |
| ASCL1 | ACHAETE-SCUTE | Central hypoventilation | AD | Hypoventilation |
| | COMPLEX, DROSOPHILA, | syndrome, congenita | | |
| | HOMOLOG OF, 1 | | | |
| ATP1A2 | ATPase, Na+/K+ | Alternating hemiplegia of | AD | Neuro |
| | TRANSPORTING, ALPHA-2 | childhood; migraine | | |
| | POLYPEPTIDE | | | |
| BAG3 | BCL2-ASSOCIATED | Cardiomyopathy, dilated, | AD | Cardiac |
| | ATHANOGENE 3 | 1HH; Myopathy, | | |
| | | myofibrillar, 6 | | |
| CACNA1A | CALCIUM CHANNEL, | Epileptic encephalopathy, | AD | Epilepsy |
| | VOLTAGE-DEPENDENT, | early infantile, 42; Episodic | | |
| | P/Q TYPE, ALPHA-1A | ataxia, type 2; Migraine, | | |
| | SUBUNIT | familial hemiplegic; | | |
| | | Spinocerebellar ataxia 6 | | |
| CACNA1C | CALCIUM CHANNEL, | Brugada syndrome 3; Timothy syndrome | | Cardiac |
| | VOLTAGE-DEPENDENT, L | | | |
| | TYPE, ALPHA-1C | | | |
| | SUBUNIT | | | |
| CACNA1H | CALCIUM CHANNEL, | {Epilepsy, childhood | AD | Epilepsy |
| | VOLTAGE-DEPENDENT, T | absence, susceptibility to, | | |
| | TYPE, ALPHA-1H SUBUNI | 6}; {Epilepsy, idiopathic | | |
| | | generalized, susceptibility | | |
| | | to, 6}; Hyperaldosteronism, | | |
| | | familial, type IV | | |
| CACNB4 | CALCIUM CHANNEL, | Episodic ataxia, type 5; | AD | Epilepsy |
| | VOLTAGE-DEPENDENT, | {Epilepsy, idiopathic | | |
| | BETA-4 SUBUNIT | generalized, susceptibility | | |
| | | to, 9}; {Epilepsy, juvenile | | |
| | | myoclonic, susceptibility to, | | |
| | | 6} | | |
| CALM1 | CALMODULIN 1 | Long QT syndrome 14; | AD | Cardiac |
| | | Ventricular tachycardia, | | |
| | | catecholaminergic | | |
| | | polymorphic, 4 | | |
| CALM2 | CALMODULIN 2 | Long QT syndrome 15 | AD | Cardiac |
| CALM3 | Calmodulin 3 | Long QT syndrome 1 and | | Cardiac |
| | | Otomycosis | | |
| CALR3 | CALRETICULIN 2 | ?Cardiomyopathy, | | Cardiac |
| | | hypertrophic, 19 | | |
| CAMK2G | CALCIUM/CALMODULIN- | Timothy Syndrome, also known as long | | Cardiac |
| | DEPENDENT PROTEIN | QT syndrome with syndactyly | | |
| | KINASE II-GAMMA | | | |
| CARS2 | CYSTEINYL-tRNA | Combined oxidative | AR | Metabolic |
| | SYNTHETASE 2 | phosphorylation deficiency | | |
| | | 27 - includes epilepsy and | | |
| | | movement disorder | | |
| CASQ2 | CALSEQUESTRIN 2 | Ventricular tachycardia, | AR | Cardiac |
| | | catecholaminergic | | |
| | | polymorphic, 2 | | |
| CAV3 | CAVEOLIN 3 | Cardiomyopathy, familial | AD | Cardiac |
| | | hypertrophic; Long QT | | |
| | | syndrome 9 | | |
| CDKL5 | CYCLIN-DEPENDENT | Epileptic encephalopathy, | XLD | Epilepsy |
| | KINASE-LIKE 5 | early infantile, 2 | | |
| CHD2 | CHROMODOMAIN | Epileptic encephalopathy, | AD | Epilepsy |
| | HELICASE DNA-BINDING | childhood-onset | | |
| | PROTEIN 2 | | | |
| CHRNA2 | CHOLINERGIC | Epilepsy, nocturnal frontal | AD | Epilepsy |
| | RECEPTOR, NEURONAL | lobe, type 4 | | |
| | NICOTINIC, ALPHA | | | |
| | POLYPEPTIDE 2 | | | |
| CHRNA3 | CHOLINERGIC | [extracellular ligand-gated ion channel | | Epilepsy |
| | RECEPTOR, NEURONAL | activity and ligand-gated ion channel | | |
| | NICOTINIC, ALPHA | activity] | | |
| | POLYPEPTIDE 3 | | | |
| CHRNA4 | CHOLINERGIC | Epilepsy, nocturnal frontal | AD | Epilepsy |
| | RECEPTOR, NEURONAL | lobe, 1 | | |
| | NICOTINIC, ALPHA | | | |
| | POLYPEPTIDE 4 | | | |
| CHRNA5 | CHOLINERGIC | [extracellular ligand-gated ion channel | | Cardiac |
| | RECEPTOR, NEURONAL | activity and ligand-gated ion channel | | |
| | NICOTINIC, ALPHA | activity] | | |
| | POLYPEPTIDE 5 | | | |
| CNTN2 | CONTACTIN 2 | ?Epilepsy, myoclonic, | AR | Epilepsy |
| | | familial adult, 5 | | |
| CNTNAP2 | CONTACTIN- | Cortical dysplasia-focal | AR | Epilepsy |
| | ASSOCIATED PROTEIN- | epilepsy syndrome; Pitt- | | |
| | LIKE 2 | Hopkins like syndrome 1 | | |
| COA5 | cytochrome c oxidase | Cardioencephalomyopathy, | AR | Cardiac |
| | assembly factor 5 | Fatal Infantile, Due to | | |
| | | Cytochrome C Oxidase | | |
| | | Deficiency 3 and Fatal | | |
| | | Infantile Cytochrome C | | |
| | | Oxidase Deficiency | | |
| CPA6 | CARBOXYPEPTIDASE A6 | Epilepsy, familial temporal | AD, AR | Epilepsy |
| | | lobe, 5; Febrile seizures, | | |
| | | familial | | |
| CPT1A | CARNITINE | CPT deficiency, hepatic, | AR | Metabolic |
| | PALMITOYLTRANSFERASE | type IA | | |
| | I, LIVER | | | |
| CSRP3 | CYSTEINE- AND | Cardiomyopathy, | AD | Cardiac |
| | GLYCINE-RICH PROTEIN | hypertrophic, 12; | | |
| | 3 | ?Cardiomyopathy, dilated, | | |
| | | 1M | | |
| CSTB | CYSTATIN B | Epilepsy, progressive | AR | Epilepsy |
| | | myoclonic 1A | | |
| CTNNA3 | catenin, cadherin-associated | Arrhythmogenic Right | AD | Cardiac |
| | protein, alpha 3 | Ventricular Dysplasia, | | |
| | | Familial, 13 and | | |
| | | Arrhythmogenic Right | | |
| | | Ventricular | | |
| | | Dysplasia/Cardiomyopathy 13 | | |
| DEPDC5 | DEP DOMAIN- | Epilepsy, familial focal, | AD | Epilepsy |
| | CONTAINING PROTEIN 5 | with variable foci 1 | | |
| DES | DESMIN | Cardiomyopathy, dilated, 1I | | Cardiac |
| DNM1 | DYNAMIN 1 | Epileptic Encephalopathy, | AD | Epilepsy |
| | | Early Infantile, 31 and | | |
| | | Lennox-Gastaut Syndrome | | |
| DOCK7 | DEDICATOR OF | Epileptic encephalopathy, | AR | Epilepsy |
| | CYTOKINESIS 7 | early infantile, 23 | | |
| DOLK | dolichol kinase | Congenital Disorder of | AR | Metabolic |
| | | Glycosylation; Familial | | |
| | | Isolated Dilated | | |
| | | Cardiomyopathy | | |
| DSC2 | DESMOCOLLIN 2 | Arrhythmogenic right | AD, AR | Cardiac |
| | | ventricular dysplasia 11 | | |
| DSG2 | DESMOGLEIN 2 | Arrhythmogenic right | AD | Cardiac |
| | | ventricular dysplasia 10; | | |
| | | Cardiomyopathy, dilated, | | |
| | | 1BB | | |
| DSP | DESMOPLAKIN | Arrhythmogenic right | AD | Cardiac |
| | | ventricular dysplasia 8; | | |
| | | Dilated cardiomyopathy | | |
| | | with woolly hair, | | |
| | | keratoderma, and tooth | | |
| | | agenesis | | |
| DTNA | dystrobrevin, alpha | Left ventricular | AD | Cardiac |
| | | noncompaction 1, with or | | |
| | | without congenital heart | | |
| | | defects | | |
| ECE1 | endothelin converting enzyme | ?Hirschsprung disease, | AD | Cardiac |
| | 1 | cardiac defects, and | | |
| | | autonomic dysfunction | | |
| EMD | EMERIN | Emery-Dreifuss muscular | XLR | Cardiac |
| | | dystrophy 1, X-linked; | | |
| | | Arrhythmogenic right | | |
| | | ventricular cardiomyopathy | | |
| | | (ARVC) (pathway) | | |
| EPM2A | EPM2A GENE | Epilepsy, Progressive | AR | Epilepsy |
| | | Myoclonic 2B and Epm2a- | | |
| | | Related Lafora Disease | | |
| ETFA | ELECTRON TRANSFER | Glutaric acidemia IIA | AR | Metabolic |
| | FLAVOPROTEIN, ALPHA | | | |
| | POLYPEPTIDE | | | |
| ETFDH | ELECTRON TRANSFER | Glutaric Acidemia IIc and | AR | Metabolic |
| | FLAVOPROTEIN | Multiple Acyl-CoA | | |
| | DEHYDROGENASE | Dehydrogenation | | |
| | | Deficiency, Severe Neonatal | | |
| | | Type | | |
| EYA4 | EYES ABSENT 4 | Cardiomyopathy, dilated, 1J | | Cardiac |
| FBN1 | FIBRILLIN 1 | Marfan syndrome | AD | Cardiac |
| FBP1 | FRUCTOSE-1,6- | Fructose-1,6-bisphosphatase | AR | Metabolic |
| | BISPHOSPHATASE 1 | deficiency | | |
| FHL2 | FOUR-AND-A-HALF LIM | Familial Isolated Dilated Cardiomyopathy | | Cardiac |
| | DOMAINS 2 | and Rhabdomyosarcoma | | |
| FKTN | FUKUTIN | Cardiomyopathy, dilated, | AR | Cardiac |
| | | 1X | | |
| FLNA | FILAMIN A | Cardiac valvular dysplasia, | AR | Cardiac |
| | | X-linked | | |
| FOXRED1 | AD-DEPENDENT | Mitochondrial Complex I | AR | Metabolic |
| | OXIDOREDUCTASE | Deficiency and Leigh | | |
| | DOMAIN-CONTAINING | Syndrome | | |
| | PROTEIN 1 | | | |
| GABRA1 | GAMMA-AMINOBUTYRIC | Epileptic encephalopathy, | AD | Epilepsy |
| | ACID RECEPTOR, ALPHA- | early infantile, 19 | | |
| | 1 | | | |
| GABRA2 | GAMMA-AMINOBUTYRIC | {Alcohol dependence, | | |
| | ACID RECEPTOR, ALPHA- | susceptibility to} | | |
| | 2 | | | |
| GABRB3 | GAMMA-AMINOBUTYRIC | Epileptic encephalopathy, | AD | Epilepsy |
| | ACID RECEPTOR, BETA-3 | early infantile, 43 | | |
| GABRD | GAMMA-AMINOBUTYRIC | Epilepsy, Generalized, With | AD | Epilepsy |
| | ACID RECEPTOR, DELTA | Febrile Seizures Plus, Type | | |
| | | 5 and Gabrd-Related | | |
| | | Generalized Epilepsy with | | |
| | | Febrile Seizures Plus | | |
| GABRG1 | GAMMA-AMINOBUTYRIC | Alcohol Dependence | | |
| | ACID RECEPTOR, | | | |
| | GAMMA-1 | | | |
| GABRG2 | GAMMA-AMINOBUTYRIC | Epilepsy, generalized, with | AD | Epilepsy |
| | ACID RECEPTOR, | febrile seizures plus, type 3; | | |
| | GAMMA-2 | Febrile seizures, familial, 8 | | |
| GAMT | CEREBRAL CREATINE | Cerebral creatine deficiency | AR | Metabolic |
| | DEFICIENCY SYNDROME | syndrome 2 | | |
| | 2 | | | |
| GATAD1 | GATA ZINC FINGER | Cardiomyopathy, Dilated, | AR | Cardiac |
| | DOMAIN-CONTAINING | 2B | | |
| | PROTEIN 1 | | | |
| GJA5 | Gap junction protein alpha 5 | Atrial Fibrillation, Familial, | AD | Cardiac |
| | | 11 and Atrial Standstill, | | |
| | | Digenic | | |
| GNAO1 | GUANINE NUCLEOTIDE- | Epileptic encephalopathy, | AD | Epilepsy |
| | BINDING PROTEIN, | early infantile, 17 | | |
| | ALPHA-ACTIVATING | | | |
| | ACTIVITY POLYPEPTIDE | | | |
| | O | | | |
| GOSR2 | GOLGI SNAP RECEPTOR | Epileptic encephalopathy, | AR | Epilepsy |
| | COMPLEX MEMBER 2 | early infantile, 17 | | |
| GPD1L | GLYCEROL-3-PHOSPHATE | Brugada syndrome 2 | | Cardiac |
| | DEHYDROGENASE 1-LIKE | | | |
| GRIN2A | GLUTAMATE RECEPTOR, | Epilepsy, focal, with speech | AD | Epilepsy |
| | IONOTROPIC, N-METHYL- | disorder and with or without | | |
| | D-ASPARTATE, SUBUNIT | mental retardation | | |
| | 2A | | | |
| HCN1 | HYPERPOLARIZATION- | Epileptic Encephalopathy, | AD | Epilepsy |
| | ACTIVATED CYCLIC | Early Infantile, 24 and | | |
| | NUCLEOTIDE-GATED | Undetermined Early-Onset | | |
| | POTASSIUM CHANNEL 1 | Epileptic Encephalopathy | | |
| HCN2 | HYPERPOLARIZATION- | Sinoatrial Node Disease. | | Cardiac |
| | ACTIVATED CYCLIC | | | |
| | NUCLEOTIDE-GATED | | | |
| | POTASSIUM CHANNEL 2 | | | |
| HCN4 | HYPERPOLARIZATION- | Brugada syndrome 8; Sick | AD | Cardiac |
| | ACTIVATED CYCLIC | sinus syndrome 2 | | |
| | NUCLEOTIDE-GATED | | | |
| | POTASSIUM CHANNEL 4 | | | |
| HEY2 | HAIRY/ENHANCER OF | Tricuspid Valve Stenosis and Tricuspid | | Cardiac |
| | SPLIT-RELATED WITH | Atresia; Common variants of SCN5A, | | |
| | YRPW MOTIF 2 | SCN10A, and HEY2 (this gene) are | | |
| | | associated with Brugada syndrome | | |
| HSD17B10 | 17-BETA- | Hsd10 Mitochondrial | XLD | Metabolic |
| (SCHAD) | HYDROXYSTEROID | Disease and 2-Methyl-3- | | |
| | DEHYDROGENASE X | Hydroxybutyric Aciduria | | |
| IFNG | INTERFERON, GAMMA | {immune modulator} | | Infectious |
| IVD | ISOVALERYL-CoA | Isovaleric acidemia | AR | Metabolic |
| | DEHYDROGENASE | | | |
| JPH2 | JUNCTOPHILIN 2 | Cardiomyopathy, | AD | Cardiac |
| | | hypertrophic, 17 | | |
| JUP | JUNCTION PLAKOGLOBIN | Arrhythmogenic right | AD | Cardiac |
| | | ventricular dysplasia 12 | | |
| KCNA1 | POTASSIUM CHANNEL, | Episodic ataxia/myokymia | AD | Cardiac/ |
| | VOLTAGE-GATED, | syndrome | | epilepsy |
| | SHAKER-RELATED | | | |
| | SUBFAMILY, MEMBER 1 | | | |
| KCNA2 | POTASSIUM CHANNEL, | Epileptic encephalopathy, | AD | Epilepsy |
| | VOLTAGE-GATED, | early infantile, 32 | | |
| | SHAKER-RELATED | | | |
| | SUBFAMILY, MEMBER 2 | | | |
| KCNB1 | POTASSIUM CHANNEL, | Epileptic Encephalopathy, | AD | Epilepsy |
| | VOLTAGE-GATED, SHAB- | Early Infantile, 26 and | | |
| | RELATED SUBFAMILY, | Undetermined Early-Onset | | |
| | MEMBER 1 | Epileptic Encephalopathy | | |
| KCNB2 | POTASSIUM CHANNEL, | Brugada Syndrome | | Cardiac |
| | VOLTAGE-GATED, SHAB- | | | |
| | RELATED SUBFAMILY, | | | |
| | MEMBER 2 | | | |
| KCND3 | POTASSIUM VOLTAGE- | Brugada syndrome 9 | AD | Cardiac |
| | GATED CHANNEL, SHAL- | | | |
| | RELATED SUBFAMILY, | | | |
| | MEMBER 3 | | | |
| KCNE1 | POTASSIUM CHANNEL, | Long QT syndrome 5 | AD | Cardiac |
| | VOLTAGE-GATED, ISK- | | | |
| | RELATED SUBFAMILY, | | | |
| | MEMBER 1 | | | |
| KCNE2 | POTASSIUM CHANNEL, | Long QT syndrome 6; | AD | Cardiac |
| | VOLTAGE-GATED, ISK- | Atrial fibrillation 4 | | |
| | RELATED SUBFAMILY, | | | |
| | MEMBER 2 | | | |
| KCNE3 | POTASSIUM CHANNEL, | Brugada syndrome 6 | | Cardiac |
| | VOLTAGE-GATED, ISK- | | | |
| | RELATED SUBFAMILY, | | | |
| | MEMBER 3 | | | |
| KCNE4 | POTASSIUM CHANNEL, | Hypokalemic Periodic Paralysis, Type 1; | | Cardiac |
| | VOLTAGE-GATED, ISK- | Antiarrhythmic Pathway | | |
| | RELATED SUBFAMILY, | | | |
| | MEMBER 4 | | | |
| KCNE1L | POTASSIUM CHANNEL, | Kcne5-Related Atrial | | Cardiac |
| (KCNE5) | VOLTAGE-GATED, ISK- | Fibrillation | | |
| | RELATED FAMILY, | | | |
| | MEMBER 1-LIKE | | | |
| KCNH2 | POTASSIUM CHANNEL, | Long QT syndrome 2; | AD | Cardiac |
| | VOLTAGE-GATED, | Short QT syndrome 1 | | |
| | SUBFAMILY H, MEMBER | | | |
| | 2 | | | |
| KCNJ3 | POTASSIUM CHANNEL, | Leber Congenital Amaurosis 16; Andersen | | Cardiac |
| | INWARDLY RECTIFYING, | Syndrome; Antiarrhythmic Pathway | | |
| | SUBFAMILY J, MEMBER 3 | | | |
| KCNJ5 | POTASSIUM CHANNEL, | Long QT syndrome 13; | | Cardiac |
| | INWARDLY RECTIFYING, | Hyperaldosteronism, familial, type III | | |
| | SUBFAMILY J, MEMBER 5 | | | |
| KCNJ8 | POTASSIUM CHANNEL, | Sudden Infant Death Syndrome; rectifying | | Cardiac |
| | INWARDLY RECTIFYING, | K+ channels; Antiarrhythmic Pathway⁷ | | |
| | SUBFAMILY J, MEMBER 8 | | | |
| KCNMA1 | POTASSIUM CHANNEL, | Paroxysmal nonkinesigenic | AD | Epilepsy |
| | CALCIUM-ACTIVATED, | dyskinesia, 3, with or | | |
| | LARGE CONDUCTANCE, | without generalized | | |
| | SUBFAMILY M, ALPHA | epilepsy | | |
| | MEMBER 1 | | | |
| KCNQ1 | POTASSIUM CHANNEL, | Long QT syndrome 1; | AD | Cardiac |
| | VOLTAGE-GATED, KQT- | Short QT syndrome 2 | | |
| | LIKE SUBFAMILY, | | | |
| | MEMBER 1 | | | |
| KCNQ2 | POTASSIUM CHANNEL, | Epileptic encephalopathy, | AD | Epilepsy |
| | VOLTAGE-GATED, KQT- | early infantile, 7 | | |
| | LIKE SUBFAMILY, | | | |
| | MEMBER 2 | | | |
| KCNQ3 | POTASSIUM CHANNEL, | Seizures, benign neonatal, | AD | Epilepsy |
| | VOLTAGE-GATED, KQT- | type 2 | | |
| | LIKE SUBFAMILY, | | | |
| | MEMBER 3 | | | |
| KCNT1 | POTASSIUM CHANNEL, | Epileptic encephalopathy, | AD | Epilepsy |
| | SUBFAMILY T, MEMBER 1 | early infantile, 14; Epilepsy, | | |
| | | nocturnal frontal lobe, 5 | | |
| KCNT2 | POTASSIUM CHANNEL, | ?Epileptic encephalopathy, | AD | Epilepsy |
| | SUBFAMILY T, MEMBER 2 | early infantile, 57 | | |
| LAMP2 | LYSOSOME-ASSOCIATED | Danon Disease (X-linked | XLD | Cardiac |
| | MEMBRANE PROTEIN 2 | vacuolar cardiomyopathy | | |
| | | and myopathy) | | |
| LDB3 | LIM DOMAIN-BINDING 3 | Cardiomyopathy, | AD | Cardiac |
| (ZASP) | | hypertrophic or dilated | | |
| LGI1 | LEUCINE-RICH GENE, | Epilepsy, familial temporal | AD | Epilepsy |
| | GLIOMA-INACTIVATED, 1 | lobe, 1 | | |
| LMNA | LAMIN A/C | Cardiomyopathy, dilated, | AD | Cardiac |
| | | 1A | | |
| MAGE | MEMBRANE- | Nephrotic Syndrome 15; Magi2-Related | | Epilepsy |
| | ASSOCIATED | Infantile Spasms | | |
| | GUANYLATE KINASE, | | | |
| | WW AND PDZ DOMAINS- | | | |
| | CONTAINING, 2 | | | |
| MBD5 | METHYL-CpG-BINDING | Mental retardation, autosomal dominant 1 | | |
| | DOMAIN PROTEIN 5 | | | |
| ME2 | MALIC ENZYME 2 | Idiopathic Generalized Epilepsy and | | Epilepsy |
| | | Generalized Epilepsy with Febrile | | |
| | | Seizures Plus | | |
| MEF2C | MADS BOX | Mental Retardation, | AD | Epilepsy |
| | TRANSCRIPTION | Stereotypic Movements, | | |
| | ENHANCER FACTOR 2, | Epilepsy, and/or Cerebral | | |
| | POLYPEPTIDE C | Malformations and | | |
| | | Arrhythmogenic Right | | |
| | | Ventricular Dysplasia 5 | | |
| MIB1 | MINDBOMB, | Left ventricular | AD | Cardiac |
| | DROSOPHILA, HOMOLOG | noncompaction 7 | | |
| | OF, 1 | | | |
| MMACHC | MMACHC GENE | Methylmalonic aciduria and | AR | Metabolic |
| | | homocystinuria, cblC type | | |
| MRPL3 | MITOCHONDRIAL | Combined Oxidative | AR | Metabolic |
| | RIBOSOMAL PROTEIN L3 | Phosphorylation Deficiency | | |
| | | 9 and Mitochondrial | | |
| | | Cardiomyopathy | | |
| MUT | METHYLMALONYL-CoA | Methylmalonic aciduria, | AR | Metabolc |
| | MUTASE | mut(0) type | | |
| MYBPC3 | MYOSIN-BINDING | Cardiomyopathy, dilated, | AD | Cardiac |
| | PROTEIN C, CARDIAC | 1MM; Cardiomyopathy, | | |
| | | hypertrophic, 4; Left | | |
| | | ventricular noncompaction | | |
| | | 10 | | |
| MYH7B | MYOSIN, HEAVY CHAIN | Left Ventricular | | Cardiac |
| | 7B, CARDIAC MUSCLE, | Noncompaction | | |
| | BETA | | | |
| MYL2 | MYOSIN, LIGHT CHAIN 2, | Cardiomyopathy, | AD | Cardiac |
| | REGULATORY, CARDIAC, | hypertrophic, 10 | | |
| | SLOW | | | |
| MYL3 | MYOSIN, LIGHT CHAIN 3, | Cardiomyopathy, | AD | Cardiac |
| | ALKALI, VENTRICULAR, | hypertrophic, 8 | | |
| | SKELETAL, SLOW | | | |
| MYL4 | MYOSIN, LIGHT CHAIN 4, | ?Atrial fibrillation, familial, | AD | Cardiac |
| | ALKALI, ATRIAL, | 18 | | |
| | EMBRYONIC | | | |
| MYLK2 | MYOSIN LIGHT CHAIN | Cardiomyopathy, | AD | Cardiac |
| | KINASE 2 | hypertrophic, 1, digenic | | |
| MΥPN | MYOPALLADIN | Cardiomyopathy, dilated, | AD | Cardiac |
| | | 1KK; Cardiomyopathy, | | |
| | | familial restrictive, 4; | | |
| | | Cardiomyopathy, | | |
| | | hypertrophic, 22 | | |
| MYOZ2 | MYOZENIN 2 | Cardiomyopathy, | AD | Cardiac |
| | | hypertrophic, 16 | | |
| NEBL | NEBULETTE | Endocardial Fibroelastosis and Dilated | | Cardiac |
| | | Cardiomyopathy | | |
| NECAP1 | NECAP ENDOCYTOSIS- | ?Epileptic encephalopathy, | AR | Epilepsy |
| | ASSOCIATED PROTEIN 1 | early infantile, 21 | | |
| NEXN | NEXILIN F-ACTIN- | Cardiomyopathy, dilated, | AD | Cardiac |
| | BINDING PROTEIN | 1CC; Cardiomyopathy, | | |
| | | hypertrophic, 20 | | |
| NHLRC1 | NHL REPEAT- | Epilepsy, progressive | AR | Epilepsy |
| | CONTAINING 1 GENE | myoclonic 2B (Lafora) | | |
| NLRP12 | NLR FAMILY, PYRIN | Familial cold | AD | Immune |
| | DOMAIN-CONTAINING 12 | autoinflammatory syndrome | | |
| | | 2 | | |
| NOS1AP | NITRIC OXIDE SYNTHASE | Sudden Infant Death Syndrome (SIDS) Susceptibility | | |
| | 1 (NEURONAL) ADAPTOR | Pathways; Circadian entrainment | | |
| | PROTEIN | | | |
| NPRL2 | NPR2-LIKE PROTEIN, | Epilepsy, familial focal, | AD | Epilepsy |
| | GATOR1 COMPLEX | with variable foci 2 | | |
| | SUBUNIT | | | |
| NPRL3 | NITROGEN PERMEASE | Epilepsy, familial focal, | AD | Epilepsy |
| | REGULATOR-LIKE 3 | with variable foci 3 | | |
| PCCA | PROPIONYL-CoA | Propionic acidemia | AR | Metabolic |
| | CARBOXYLASE, ALPHA | | | |
| | SUBUNIT | | | |
| PCCB | PROPIONYL-CoA | Propionic acidemia | AR | Metabolic |
| | CARBOXYLASE, BETA | | | |
| | SUBUNIT | | | |
| PCDH19 | PROTOCADHERIN 19 | Epileptic encephalopathy, | XL | Epilepsy |
| | | early infantile, 9 | | |
| PHOX2B | PAIRED-LIKE HOMEOBOX | Central hypoventilation | AD | Hypoventilation |
| | 2B | syndrome, congenital, with | | |
| | | or without Hirschsprung | | |
| | | disease | | |
| PKP2 | PLAKOPHILIN 2 | Arrhythmogenic right | AD | Cardiac |
| | | ventricular dysplasia 9 | | |
| PLCB1 | PHOSPHOLIPASE C, | Epileptic encephalopathy, | AR | Epilepsy |
| | BETA-1 | early infantile, 12 | | |
| PLN | PHOSPHOLAMB AN | Cardiomyopathy, dilated, | AD | Cardiac |
| | | 1P; Cardiomyopathy, | | |
| | | hypertrophic, 18 | | |
| PNKP | POLYNUCLEOTIDE | Microcephaly, seizures, and | AR | Epilepsy |
| | KINASE 3-PRIME | developmental delay | | |
| | PHOSPHATASE | | | |
| PNPO | PYRIDOXAMINE 5-PRIME- | Pyridoxamine 5′-phosphate | AR | Metabolic |
| | PHOSPHATE OXIDASE | oxidase deficiency | | |
| PRICKLE1 | PRICKLE, DROSOPHILA, | Epilepsy, progressive | AR | Epilepsy |
| | HOMOLOG OF, 1 | myoclonic 1B | | |
| PRICKLE2 | PRICKLE, DROSOPHILA, | Progressive Myoclonic | | Epilepsy |
| | HOMOLOG OF, 2 | Epilepsy 5 | | |
| PRKAG2 | PROTEIN KINASE, AMP- | Cardiomyopathy, | AD | Cardiac |
| | ACTIVATED, | hypertrophic 6; Wolff- | | |
| | NONCATALYTIC, | Parkinson-White syndrome | | |
| | GAMMA-2 | | | |
| PRRT2 | PROLINE-RICH | Seizures, benign familial | AD | Epilepsy |
| | TRANSMEMBRANE | infantile, 2; Episodic | | |
| | PROTEIN 2 | kinesigenic dyskinesia 1; | | |
| | | Convulsions, familial | | |
| | | infantile, with paroxysmal | | |
| | | choreoathetosis | | |
| PSEN1 | PRESENILIN 1 | Cardiomyopathy, dilated, | AD | Cardiac |
| | | 1U; Dementia, | | |
| | | Frontotemporal and | | |
| | | Alzheimer Disease, Type 3 | | |
| PSEN2 | PRESENILIN 2 | Cardiomyopathy, dilated, | AD | Cardiac |
| | | 1V; Alzheimer disease-4 | | |
| PYGM | GLYCOGEN | McArdle disease | AR | Metabolic |
| | PHOSPHORYLASE, | | | |
| | MUSCLE | | | |
| RAF1 | V-RAF-1 MURINE | Cardiomy opathy, dilated, | AD | Cardiac |
| | LEUKEMIA VIRAL | 1NN; LEOPARD syndrome | | |
| | ONCOGENE HOMOLOG 1 | 2; Noonan syndrome | | |
| RANBP2 | RAN-BINDING PROTEIN 2 | {Encephalopathy, acute, | AD | Infectious |
| | | infection-induced, 3, | | |
| | | susceptibility to} | | |
| RANGRF | RAN GUANINE | Brugada Syndrome | | Cardiac |
| | NUCLEOTIDE RELEASE | | | |
| | FACTOR | | | |
| RBM20 | RNA-BINDING MOTIF | Cardiomyopathy, dilated, | AD | Cardiac |
| | PROTEIN 20 | 1DD | | |
| SCARB2 | SCAVENGER RECEPTOR | Epilepsy, progressive | AR | Epilepsy |
| | CLASS B, MEMBER 2 | myoclonic 4, with or | | |
| | | without renal failure | | |
| SCN1A | SODIUM CHANNEL, | Epilepsy, generalized, with | AD | Epilepsy |
| | NEURONAL TYPE I, | febrile seizures plus, type 2; | | |
| | ALPHA SUBUNIT | Febrile seizures, familial, | | |
| | | 3A | | |
| SCN1B | SODIUM CHANNEL, | Epilepsy, generalized, with | AD | Cardiac/ |
| | VOLTAGE-GATED, TYPE I, | febrile seizures plus, type 1; | | Epilepsy |
| | BETA SUBUNIT | Brugada syndrome 5; Atrial | | |
| | | fibrillation, familial, 13 | | |
| SCN2A | SODIUM CHANNEL, | Epileptic encephalopathy, | AD | Epilepsy |
| | VOLTAGE-GATED, TYPE | early infantile, 11; Seizures, | | |
| | II, ALPHA SUBUNIT | benign familial infantile, 3 | | |
| SCN2B | SODIUM CHANNEL, | Atrial Fibrillation, Familial, | AD | Cardiac |
| | VOLTAGE-GATED, TYPE | 14 | | |
| | II, BETA SUBUNIT | | | |
| SCN3A | SODIUM CHANNEL, | Scn3a-Related Focal Epilepsy | | Epilepsy |
| | VOLTAGE-GATED, TYPE | Susceptibility | | |
| | III, ALPHA SUBUNIT | | | |
| SCN3B | SODIUM CHANNEL, | Atrial fibrillation, familial, | AD | Cardiac |
| | VOLTAGE-GATED, TYPE | 16; Brugada syndrome 7 | | |
| | III, BETA SUBUNIT | | | |
| SCN7A | SODIUM CHANNEL, | Normokalemic Periodic Paralysis and | | Neuro |
| | VOLTAGE-GATED, TYPE | Hypokalemic Periodic Paralysis, Type 2 | | |
| | VII, ALPHA SUBUNIT | | | |
| SCN8A | SODIUM CHANNEL, | Epileptic encephalopathy, | AD | Epilepsy |
| | VOLTAGE-GATED, TYPE | early infantile, 13; Seizures, | | |
| | VIII, ALPHA SUBUNIT | benign familial infantile, 5 | | |
| SCN11A | SODIUM CHANNEL, | Episodic pain syndrome, familial, 3; | | Neuro |
| | VOLTAGE-GATED, TYPE | Neuropathy, hereditary sensory and | | |
| | XI, ALPHA SUBUNIT | autonomic, type VII | | |
| SCO2 | SCO2 CYTOCHROME c | Cardioencephalomyopathy, | AR | Cardiac/ |
| | OXIDASE ASSEMBLY | Fatal Infantile, Due to | | Epilepsy |
| | PROTEIN | Cytochrome C Oxidase | | |
| | | Deficiency 1 | | |
| SGCD | SARCOGLYCAN, DELTA | Cardiomyopathy, dilated, | | Cardiac |
| | | 1L | | |
| SLC35A2 | SOLUTE CARRIER | Congenital disorder of | XLD, | Metabolic |
| | FAMILY 35 (UDP- | glycosylation, type IIm | Somatic | |
| | GALACTOSE | | mosaicism | |
| | TRANSPORTER), MEMBER | | | |
| | 2 | | | |
| SLC25A4 | SOLUTE CARRIER | Mitochondrial DNA | AD, AR | Cardiac/ |
| | FAMILY 25 | depletion syndrome 12A | | Metabolic |
| | (MITOCHONDRIAL | (cardiomyopathic type) AD | | |
| | CARRIER, ADENINE | | | |
| | NUCLEOTIDE | | | |
| | TRANSLOCATOR), | | | |
| | MEMBER 4 | | | |
| SLC25A22 | SOLUTE CARRIER | Epileptic encephalopathy, | AR | Epilepsy |
| | FAMILY 25 | early infantile, 3 | | |
| | (MITOCHONDRIAL | | | |
| | CARRIER, GLUTAMATE), | | | |
| | MEMBER 22 | | | |
| SLC2A1 | SOLUTE CARRIER | {Epilepsy, idiopathic | AD | Epilepsy |
| | FAMILY 2 (FACILITATED | generalized, susceptibility | | |
| | GLUCOSE | to, 12}; GLUT1 deficiency | | |
| | TRANSPORTER), MEMBER | syndrome | | |
| | 1 | | | |
| SLC2A2 | SOLUTE CARRIER | Fanconi-Bickel syndrome | AR | Metabolic |
| | FAMILY 2 (FACILITATED | (glycogen storage disease) | | |
| | GLUCOSE | | | |
| | TRANSPORTER), MEMBER | | | |
| | 2 | | | |
| SLC6A1 | SOLUTE CARRIER | Myoclonic-Atonic Epilepsy and | | Epilepsy |
| | FAMILY 6 | Myoclonic-Astastic Epilepsy | | |
| | (NEUROTRANSMITTER | | | |
| | TRANSPORTER, GABA), | | | |
| | MEMBER 1 | | | |
| SLC6A4 | SOLUTE CARRIER | {Anxiety-related personality traits}; | | Metabolic |
| | FAMILY 6 | {Obsessive-compulsive disorder} | | |
| | (NEUROTRANSMITTER | | | |
| | TRANSPORTER, | | | |
| | SEROTONIN), MEMBER 4 | | | |
| SLC6A5 | SOLUTE CARRIER | Hyperekplexia 3 | AD, AR | Neuro |
| | FAMILY 6 | | | |
| | (NEUROTRANSMITTER | | | |
| | TRANSPORTER, | | | |
| | GLYCINE), MEMBER 5 | | | |
| SLC22A5 | SOLUTE CARRIER | Carnitine deficiency, | AR | Metabolic |
| | FAMILY 22 (ORGANIC | systemic primary | | |
| | CATION TRANSPORTER), | | | |
| | MEMBER 5 | | | |
| SLC22A6 | SOLUTE CARRIER | N-Acetylglutamate Synthase Deficiency; | | Metabolic |
| | FAMILY 22 (ORGANIC | Uricosurics Pathway, Pharmacodynamics | | |
| | ANION TRANSPORTER), | and Transport of glucose and other sugars, | | |
| | MEMBER 6 | bile salts and organic acids, metal ions and | | |
| | | amine compounds. | | |
| SNTA1 | SYNTROPHIN, ALPHA-1 | Long QT syndrome 12 | AD | Cardiac |
| SPTAN1 | SPECTRIN, ALPHA, | Epileptic encephalopathy, | AD | Epilepsy |
| | NONERYTHROCYTIC 1 | early infantile, 5 | | |
| ST3GAL3 | ST3 BETA-GALACTOSIDE | ?Epileptic encephalopathy, | AR | Epilepsy |
| | ALPHA-2,3- | early infantile, 15 | | |
| | SIALYLTRANSFERASE 3 | | | |
| STXBP1 | SYNTAXIN-BINDING | Epileptic encephalopathy, early infantile, 4 | | |
| | PROTEIN 1 | | | |
| SYN1 | SYNAPSIN I | Epilepsy, X-Linked, With | XLD, XLR | Epilepsy |
| | | Variable Learning | | |
| | | Disabilities and Behavior | | |
| | | Disorders and Rett | | |
| | | Syndrome | | |
| SZT2 | SEIZURE THRESHOLD 2, | Epileptic encephalopathy, | AR | Epilepsy |
| | MOUSE, HOMOLOG OF | early infantile, 18 | | |
| TAZ | TAFAZZIN | Barth syndrome (dilated | XLR | Cardiac |
| | | cardiomyopathy with | | |
| | | endocardial fibroelastosis) | | |
| TBC1D24 | TBC1 DOMAIN FAMILY, | Epileptic encephalopathy, | AR | Epilepsy |
| | MEMBER 24 | early infantile, 16; | | |
| | | Myoclonic epilepsy, | | |
| | | infantile, familial | | |
| TCAP | TITIN-CAP | Cardiomyopathy, | AD | Cardiac |
| | | hypertrophic, 25 | | |
| TGFB3 | TRANSFORMING | Arrhythmogenic right | AD | Cardiac |
| | GROWTH FACTOR, BETA- | ventricular dysplasia 1; | | |
| | 3 | Loeys-Dietz syndrome 5 | | |
| TLX3 | T-CELL LEUKEMIA | Sudden Infant Death Syndrome (SIDS) | | SIDS |
| | HOMEOBOX 3 | Susceptibility Pathway | | |
| TMPO | THYMOPOIETIN | Tmpo-Related Dilated Cardiomyopathy | | Cardiac |
| | | and Familial Isolated Dilated | | |
| | | Cardiomyopathy | | |
| TNF | TUMOR NECROSIS | {Septic shock, susceptibility | | Infectious |
| | FACTOR | to} | | |
| TNK2 | TYROSINE KINASE, | Infantile-Onset Mesial Temporal Lobe | | Epilepsy |
| | NONRECEPTOR, 2 | Epilepsy with Severe Cognitive | | |
| | | Regression. | | |
| TNNC1 | TROPONIN C, SLOW | Cardiomyopathy, dilated, | AD | Cardiac |
| | | 1Z; Cardiomyopathy, | | |
| | | hypertrophic, 13 | | |
| TNNI3 | TROPONIN I, CARDIAC | Cardiomyopathy, dilated, 2A; Cardiomyopathy, | | |
| | | familial restrictive, 1; Cardiomyopathy, hypertrophic, | | |
| | | 7 | | |
| TNNT2 | TROPONIN T2, CARDIAC | Cardiomyopathy, dilated, 1D; Cardiomyopathy, | | |
| | | dilated, 1D 601494 AD 3 | | |
| | | Cardiomyopathy, familial restrictive; | | |
| | | Cardiomyopathy, hypertrophic, 2 | | |
| TRPM4 | TRANSIENT RECEPTOR | Progressive familial heart | AD | Cardiac |
| | POTENTIAL CATION | block, type IB | | |
| | CHANNEL, SUBFAMILY | | | |
| | M, MEMBER 4 | | | |
| TRDN | TRIADIN | Ventricular tachycardia, | AR | Cardiac |
| | | catecholaminergic | | |
| | | polymorphic, 5, with or | | |
| | | without muscle weakness | | |
| TSC1 | TSC1 GENE | Tuberous sclerosis-1; Focal | AD | Epilepsy |
| | | cortical dysplasia, type II, | | |
| | | somatic | | |
| TSC2 | TSC2 GENE | Tuberous sclerosis-2; | AD | Epilepsy |
| | | ?Focal cortical dysplasia, | | |
| | | type II, somatic | | |
| TSFM | Ts TRANSLATION | Combined Oxidative | AR | Cardiac |
| | ELONGATION FACTOR, | Phosphorylation Deficiency | | |
| | MITOCHONDRIAL | 3 and Dilated | | |
| | | Cardiomyopathy | | |
| TTN | TITIN | Cardiomyopathy, dilated, | AD | Cardiac |
| | | 1G; Cardiomyopathy, | | |
| | | familial hypertrophic, 9 | | |
| TTR | TRANSTHYRETIN | Amyloidosis, Hereditary, Transthyretin-Related and | | |
| | | Carpal Tunnel Syndrome, Familial. | | |
| TXNRD2 | THIOREDOXIN | Familial Glucocorticoid Deficiency and | | Cardiac |
| | REDUCTASE 2 | Familial Isolated Dilated Cardiomyopathy | | |
| VCL | VINCULIN | Cardiomyopathy, dilated, | AD | Cardiac |
| | | 1W; Cardiomyopathy, | | |
| | | hypertrophic, 15 | | |
| LDB3 | LIM DOMAIN-BINDING 3 | Cardiomyopathy, | AD | Cardiac |
| (ZASP) | | hypertrophic or dilated | | |

| | | |
|---|---|---|
| ZEB2 | ZINC FINGER E BOX- | Mowat-Wilson Syndrome and Mowat-Wilson |
| | BINDING HOMEOBOX 2 | Syndrome Due to A Zeb2 Point Mutation |

In Table 1, QT Syndrome genes are identified in bold. Pediatric subjects having alterations in such genes are at risk for SUDP and are selected using a method of the invention for treatment with a beta blocker.

TABLE 2
SUDP Genes

Gene
Symbol
ACADM
ACADVL
ACTA1
ACTC1
ALDH7A1
ALDOB
ANKRD1
ARIX
ARX
ASCL1
ATP1A2
BAG3
CACNA1A
CACNA1H
CACNB4
CAMK2G
CARS2
CDKL5
CHD2
CHRNA2
CHRNA3
CHRNA4
CHRNA5
CNTN2
CNTNAP2
COA5
CPA6
CPT1A
CSTB
CTNNA3
DEPDC5
DNM1
DOCK7
DOLK
ECE1
EMD
EPM2A
ETFA
ETFDH
FHL2
FLNA
FOXRED1
GABRA1
GABRA2
GABRB3
GABRD
GABRG1
GABRG2
GAMT
GATAD1
GNAO1
GOSR2
GRIN2A
HCN1
HCN2
HEY2
HSD17B10
IFNG
IVD
KCNA1
KCNA2
KCNB1
KCNB2
KCNE4
KCNE1L
KCNJ3
KCNJ5
KCNMA1
KCNQ2
KCNQ3
KCNT1
KCNT2
LGI1
MAGI2
MBD5
ME2
MEF2C
MIB1
MMACHC
MRPL3
MUT
MYH7B
MYL4
MYPN
NEBL
NECAP1
NHLRC1
NLRP12
NOS1AP
NPRL2
NPRL3
PCCA
PCCB
PCDH19
PHOX2B
PLCB1
PNKP
PNPO
PRICKLE1
PRICKLE2
PRRT2
PYGM
RAF1
RANGRF
RHOBTB2
SCARB2
SCN1A
SCN2A
SCN3A
SCN7A
SCN8A
SCN11A
SCO2
SLC35A2
SLC25A22
SLC2A1
SLC2A2
SLC6A1
SLC6A4
SLC6A5
SLC22A5
SLC22A6
SPTAN1
ST3GAL3
STXBP1
SYN1
SZT2
TBC1D24
TLX3
TNF
TNK2
TRDN
TSC1
TSC2
TSFM
TXNRD2
LDB3
ZEB2

### Biologic Samples

Samples for use with in the methods of the invention include biological samples that contain nucleic acid molecules. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva. In one particular embodiment, the biological sample is brain or spleen.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a specimen or portion thereof.

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Whatever the source of the biological sample, the target polynucleotide (“Target”) that is ultimately assayed can be prepared synthetically (in the case of control sequences), but typically is purified from the biological source and subjected to one or more preparative steps. The RNA or DNA may be purified to remove or diminish one or more undesired components from the biological sample or to concentrate it. Conversely, where the RNA or DNA is too concentrated for the particular assay, it may be diluted.

### Characterization of SUDP Genes

Any method of detecting and/or quantitating a SUDP gene can in principle be used in the invention. The SUDP gene or a fragment thereof can be directly characterized (e.g., sequenced, detected and/or quantitated), or may be copied and/or amplified to allow characterization of amplified copies of the SUDP gene, expressed target sequence or its complement.

In one embodiment, SUDP genes are characterized by sequencing. In particular embodiments, exomes of a SUDP gene present in a biological sample derived from a subject are sequenced and the sequence is compared to a reference sequence that is a wild-type sequence present in NCBI or another database. In another embodiment, a trio of sequences are compared, i.e., sequences from proband and parents are compared to each other and to a reference.

Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

Methods for characterizing (e.g., detecting and/or quantifying) a SUDP gene or fragment thereof can also include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

Still other methods and compositions for gene expression is characterizing using Nanostring technology, RNAseq, or an Affymetrix-expression array.

### Probes/Primers

The present invention provides for probe sets for characterizing one or more SUDP genes of Table 1 or Table 2 using a plurality of probes, wherein (i) the probes in the set are capable of detecting an expression level of at least one target; and (ii) the expression level determines the status of the subject.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

One skilled in the art understands that the nucleotide sequence of the polynucleotide probe need not be 100% complementary to its target sequence in order to specifically hybridize thereto. The polynucleotide probes of the present invention, therefore, comprise a nucleotide sequence that is at least about 85%, 90%, or 95% complementary to a region of the coding target or non-coding target selected from those listed herein.

Methods of determining sequence identity are known in the art and can be determined, for example, by using the BLASTN program of the University of Wisconsin Computer Group (GCG) software or provided on the NCBI website. The nucleotide sequence of the polynucleotide probes of the present invention may exhibit variability by differing (e.g. by nucleotide substitution, including transition or transversion) at one, two, three, four or more nucleotides from the sequence of the coding target or non-coding target.

Other criteria known in the art may be employed in the design of the polynucleotide probes of the present invention. For example, the probes can be designed to have <50% G content and/or between about 25% and about 70% G+C content. Strategies to optimize probe hybridization to the target nucleic acid sequence can also be included in the process of probe selection.

Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus, the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to faun a linear polymeric compound, with the normal linkage or backbone of RNA and DNA being a 3′ to 5′ phosphodiester linkage. Specific examples of polynucleotide probes or primers useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide probes or primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkylphosphonates including 3′-alkylene phosphonates and chiral phosphonates, phosphinates, phosphoramidates including 3′ amino phosphoramidate and aminoalkylphosphoramidates, thionophosphoramidates, thionoalkylphosphonates, thionoalkyl phosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid fauns are also included.

Other modifications may also be made at other positions on the polynucleotide probes or primers, particularly the 3′ position of the sugar on the 3′ terminal nucleotide or in 2′-5′ linked oligonucleotides and the 5′ position of 5′ terminal nucleotide. Polynucleotide probes or primers may also have sugar mimetics such as cyclobutyl moieties in place of the pentofuranosyl sugar.

Polynucleotide probes or primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia of Polymer Science and Engineering, (1990) pp 858-859, Kroschwitz, J. L, ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability.

One skilled in the art recognizes that it is not necessary for all positions in a given polynucleotide probe or primer to be uniformly modified. The present invention, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences which are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

The polynucleotide probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target sequence is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Polynucleotides from the described target sequences may be employed as probes for detecting target sequences expression, for ligation amplification schemes, or may be used as primers for amplification schemes of all or a portion of a target sequences. When amplified, either strand produced by amplification may be provided in purified and/or isolated form.

Complements may take any polymeric form capable of base pairing to the species recited in (a)-(e), including nucleic acid such as RNA or DNA, or may be a neutral polymer such as a peptide nucleic acid. Polynucleotides of the invention can be selected from the subsets of the recited nucleic acids described herein, as well as their complements.

The polynucleotides may be provided in a variety of formats, including as solids, in solution, or in an array. The polynucleotides may optionally comprise one or more labels, which may be chemically and/or enzymatically incorporated into the polynucleotide.

In one embodiment, solutions comprising polynucleotide and a solvent are also provided. In some embodiments, the solvent may be water or may be predominantly aqueous. In some embodiments, the solution may comprise at least two, three, four, five, six, seven, eight, nine, ten, twelve, fifteen, seventeen, twenty or more different polynucleotides, including primers and primer pairs, of the invention. Additional substances may be included in the solution, alone or in combination, including one or more labels, additional solvents, buffers, biomolecules, polynucleotides, and one or more enzymes useful for performing methods described herein, including polymerases and ligases. The solution may further comprise a primer or primer pair capable of amplifying a polynucleotide of the invention present in the solution.

### SUDP Panels

In some embodiments, one or more polynucleotides (e.g., genes, fragments thereof, primers, probes) provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenediflumide, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like, and include semiconductor nanocrystals.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

Silica aerogels can also be used as substrates, and can be prepared by methods known in the art. Aerogel substrates may be used as free-standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. The surface can be optically transparent and can have surface Si—OH functionalities, such as those found on silica surfaces.

The substrate and/or its optional surface can be chosen to provide appropriate characteristics for the synthetic and/or detection methods used. The substrate and/or surface can be transparent to allow the exposure of the substrate by light applied from multiple directions. The substrate and/or surface may be provided with reflective “mirror” structures to increase the recovery of light.

The substrate and/or its surface is generally resistant to, or is treated to resist, the conditions to which it is to be exposed in use, and can be optionally treated to remove any resistant material after exposure to such conditions. The substrate or a region thereof may be encoded so that the identity of the sensor located in the substrate or region being queried may be determined. Any suitable coding scheme can be used, for example optical codes, RFID tags, magnetic codes, physical codes, fluorescent codes, and combinations of codes.

### Preparation of Probes and Primers

A marker of the invention is analyzed using a probe or primer that targets that marker. The polynucleotide probes or primers of the present invention can be prepared by conventional techniques well-known to those skilled in the art. For example, the polynucleotide probes can be prepared using solid-phase synthesis using commercially available equipment. As is well-known in the art, modified oligonucleotides can also be readily prepared by similar methods. The polynucleotide probes can also be synthesized directly on a solid support according to methods standard in the art. This method of synthesizing polynucleotides is particularly useful when the polynucleotide probes are part of a nucleic acid array.

Polynucleotide probes or primers can be fabricated on or attached to the substrate by any suitable method, for example the methods described in U.S. Pat. No. 5,143,854, PCT Publ. No. WO 92/10092, U.S. patent application Ser. No. 07/624,120, filed Dec. 6, 1990 (now abandoned), Fodor et al., Science, 251: 767-777 (1991), and PCT Publ. No. WO 90/15070). Techniques for the synthesis of these arrays using mechanical synthesis strategies are described in, e.g., PCT Publication No. WO 93/09668 and U.S. Pat. No. 5,384,261. Still further techniques include bead based techniques such as those described in PCT Appl. No. PCT/US93/04145 and pin based methods such as those described in U.S. Pat. No. 5,288,514. Additional flow channel or spotting methods applicable to attachment of sensor polynucleotides to a substrate are described in U.S. patent application Ser. No. 07/980,523, filed Nov. 20, 1992, and U.S. Pat. No. 5,384,261.

Alternatively, the polynucleotide probes of the present invention can be prepared by enzymatic digestion of the naturally occurring target gene, or mRNA or cDNA derived therefrom, by methods known in the art.

### Coding and Non-Coding Targets

The methods disclosed include assaying the sequence or expression level of a plurality of SUDP genes. The SUDP genes may comprise coding targets and/or non-coding targets subject to analysis. A protein-coding SUDP gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some embodiments, the plurality of SUDP genes may be differentially expressed. In some embodiments, the plurality of SUDP genes is selected from those listed in Table 1 or Table 2. In particular embodiments, the targets comprise coding or non-coding targets of a SUDP gene of Table 1 or Table 2. In some embodiments, the plurality of SUDP comprises at least about 2, at least about 3, at least about 4, at least about 5, at least about 6, at least about 7, at least about 8, at least about 9, or at least about 10 SUDP genes selected from those presented herein. In other instances, the plurality of SUDP genes comprises at least about 12, at least about 15, at least about 17, at least about 20, at least about 22, at least about 25, at least about 27, at least about 30, at least about 32, at least about 35, at least about 37, or at least about 40 targets selected from those listed in Table 1 or Table 2. In other instances, the plurality of SUDP genes comprises at least about 50, 60, 70, 80, 90, 100, 125, 150, or 200 SUDP genes or fragments or portions thereof for analysis.

In some instances, the plurality of SUDP genes comprises a coding target, non-coding target, or any combination thereof. In some instances, the coding target comprises an exonic sequence. In other instances, the non-coding target comprises a non-exonic sequence. Alternatively, a non-coding target comprises a UTR sequence, an intronic sequence, or a non-coding RNA transcript. In some instances, a non-coding target comprises sequences which partially overlap with a UTR sequence or an intronic sequence. A non-coding target also includes non-exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

### Amplification and Hybridization

Following sample collection and nucleic acid extraction, the nucleic acid portion of the sample comprising RNA or DNA that is or can be used to prepare the target polynucleotide(s) for analysis can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, and in many cases produces multiple copies of a polynucleotide of interest. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self sustained sequence replication (3 SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically fauns a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to a substrate (e.g., an array or microarray), a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation'm FFPE Exon Module, WT-Ovation™ FFPE Exon Module RiboAmp and RiboAmp.sup.Plus RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

### Selection of Subjects

Pediatric probands who have undergone a sudden unexpected death (i.e., a natural death) or have experienced an apparently life-threatening event (e.g., medical event, such as a respiratory, coronary or neurological event, that in the absence of medical intervention would result in death) are analyzed. The identification of one or more alterations in the sequence of a SUDP gene present in a biological sample derived from the proband indicates that the death or near-death event is associated with the alteration in the SUDP gene. The characterization of such mutation indicates that first and second-degree relatives of the proband should be selected for characterization of the SUDP gene altered in the proband. Identification of a SUDP gene sequence alteration in a relative of the proband indicates that that relative is also at risk for SUDP or is at risk for having a child that undergoes SUDP. Factors known in the art for diagnosing and/or suggesting, selecting, designating, recommending or otherwise determining a course of treatment for a patient or class of patients suspected of being at risk for SUDP can be employed in combination with characterization of the SUDP target sequence. The methods disclosed herein may include additional techniques such as neurological, metabolic, and cardiac function testing, cytology, immunocytochemistry, cardiograms, echo, histology, ultrasound analysis, MRI results, CT scan results, and measurements of other biomarker levels, patient medical history.

Certified tests for classifying disease status and/or designating treatment modalities may also be used in diagnosing, predicting, and/or monitoring the status or outcome of SUDP in a subject. A certified test may comprise a means for characterizing the sequence or expression levels of one or more of the target sequences of interest, and a certification from a government regulatory agency endorsing use of the test for classifying the disease status of a biological sample.

In some embodiments, the certified test may comprise reagents for amplification reactions used to detect and/or quantitate expression of the target sequences to be characterized in the test. An array of probe nucleic acids can be used, with or without prior target amplification, for use in characterizing a target sequence or measuring target sequence expression.

The test is submitted to an agency having authority to certify the test for use in distinguishing disease status and/or outcome. Detection of sequence alterations or alterations in expression levels of the target sequences used in the test and correlation with disease status and/or outcome are submitted to the agency. A certification authorizing the diagnostic and/or prognostic use of the test is obtained.

### Genome Analysis

Genome characterization is carried out for subjects whose deaths are characterized using SUDP gene panels of the invention. In particular, exome sequencing is carried out on polynucleotides contained in biological samples of a SUDP subject or relative thereof. Genome analysis is then carried out as follows:
1. Utilizing software to perform genome analysis to filter and identify variants using the defined gene lists provided at Table 1 or 2 or subsets of such genes. Extensive phenotype and pedigree data will be uploaded, as will the variant list, for genomic analysis of this prevalent, lethal phenotype. Seqr filtering will facilitate gene discovery and the elaboration of unrecognized genetic mechanisms.
2. Software analysis to identify potential causative variations in the untranslated regions, indels, and structural rearrangements.
3. “Matchmaker Exchange” programs and other discovery tools to identify additional cases with candidate genes of interest.
4. Pathway analysis will be used to assess bioinformatics, the effects of indels and single nucleotide polymorphisms (SNPs) in genes within candidate pathways (e.g., serotonin, potassium and sodium channels) in addition to analyzing the effects of gene-gene interactions on complex diseases.

### Reports

A patient report is also provided comprising a representation of sequence alterations or measured expression levels of a plurality of target sequences in a biological sample from the patient, wherein the representation comprises target sequences corresponding to any one, two, three, four, five, six, eight, ten, twenty, thirty, fifty, 75, 100 or more of the target sequences corresponding to SUDP genes present in Table 1 or 2, or of the subsets described herein, or of a combination thereof. The patient report may be provided in a machine (e.g., a computer) readable format and/or in a hard (paper) copy. The report can be used to inform the patient and/or treating physician of the alterations present in the target sequences, the likely medical diagnosis and/or implications, and optionally may recommend a treatment modality or monitoring for the patient.

Also provided are representations of the results of SUDP gene analyses useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene sequence in such media. For example, the articles may comprise a readable storage form having computer instructions for comparing sequences of SUDP genes described above. The articles may also have SUDP gene sequences digitally recorded therein so that they may be compared with SUDP gene sequences derived from patient samples.

### Kits

Kits for characterizing SUDP genes of the invention are also provided, and comprise a container or housing for holding the components of the kit, one or more vessels containing one or more nucleic acid(s), and optionally one or more vessels containing one or more reagents. In one embodiment, the kit includes a panel of SUDP genes of Table 1 or 2, and/or primers and/or probes that hybridize to the SUDP genes. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for one or more SUDP genes, subsets and combinations of target sequences described herein. At least two, three, four or five primers or pairs of primers suitable for selectively amplifying the same number of target sequence-specific polynucleotides can be provided in kit form. In some embodiments, the kit comprises from five to fifty, fifty to 100, or 100 to 200 or more primers or pairs of primers suitable for amplifying or otherwise characterizing the same number of target sequence-representative polynucleotides of interest. In some embodiments, the primers or primer pairs of the kit, when used in an amplification reaction, specifically amplify a non-coding target, coding target, or non-exonic target described herein.

The reagents may independently be in liquid or solid form. The reagents may be provided in mixtures. Control samples and/or nucleic acids may optionally be provided in the kit. Control samples may include tissue and/or nucleic acids obtained from or representative of samples from patients showing no evidence of disease, as well as tissue and/or nucleic acids obtained from or representative of samples from patients that develop SUDP.

The nucleic acids may be provided in an array format, and thus an array or microarray may be included in the kit. The kit optionally may be certified by a government agency for use in characterizing the disease outcome or death of SUDP patients and/or for designating a treatment or monitoring modality for first or second-degree relatives of such patients.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Identifying Underlying Diseases that Cause Sudden Unexpected Death in Pediatrics

Sudden Unexpected Death in Pediatrics (SUDP) is a leading cause of death in children under the age of 3 years, accounting for more deaths per year in the United States than childhood cancer or heart disease. A seemingly well child is discovered dead after a sleep period. In most cases SUDP remains unexplained, contributing to uncertainty and isolation in parents following their tragic loss. SUDP encompasses Sudden Infant Death Syndrome (SIDS) and Sudden Unexplained Death in Childhood (SUDC), affecting children under and over the age 1 year, respectively.

Although there is a defined age distinction with SIDS in infants under 1 year of age and SUDC in children over 1 year of age that reflects the presumption that different risk factors predict different etiologies in these age groups, the present findings indicate that SIDS and SUDC represent a continuum across the age ranges in many cases. This continuum encompasses critical periods linked to brain, cardiac, and autonomic nervous system development, perhaps with different age-dependent risk factors based on expression of genes involved in the maturation of these systems. The etiologic model for SUDP is the Triple-Risk Model of SIDS, which maintains that sudden death occurs due to a combination of latent biological vulnerabilities (e.g., genetic factors) and external factors (e.g., sleep environment) during key developmental stages of enhanced susceptibility.

Improved mortality rates in SIDS, the major component of SUDP mortality, are conventionally attributed to changes in infant sleep practices. As external factors have been addressed, the persistence of SUDP attests to the significance of intrinsic vulnerabilities. Moreover, declines in SIDS rates are largely identical to declines observed in non-SUDP causes of death, suggesting the common influence of biomedical factors responsible for decreased mortality in known diseases, including preventive care, the use of antenatal steroids, lower maternal smoking rates, and better prenatal care. Despite improvements in mortality rates, however, SUDP remains a prevalent cause of death under the age of 3 years.

Extensive studies identified biological vulnerabilities in SUDP. These studies indicated common neuropathological entities across these operationally defined syndromes, moving the field beyond associations with sleep period and being discovered prone. Strategies for detailed phenotyping were developed, including a scale to assess the risk of asphyxia contributing to death. Brainstem serotonergic abnormalities were found in about 40% of SIDS cases, and functionally demonstrated in mouse models of deficient autonomic homeostasis and seizures. Neuropathological abnormalities in the hippocampus, previously described in epilepsy, were reported in 41% of SIDS and 48% of SUDC. Such lesions are shared across the age ranges of SIDS and SUDC, suggesting a pathologic entity involving the temporal lobe underlying a large subset of SUDP.

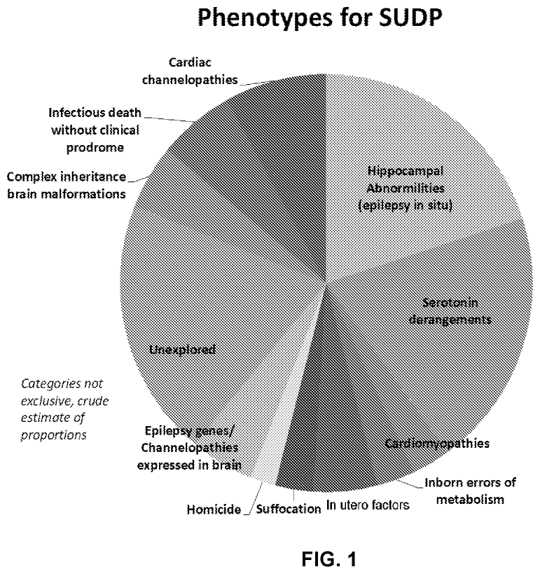

The intrinsic biological factors leading to SUDP include neurodevelopmental, epilepsy-related, cardiac, metabolic, respiratory, and infectious mechanisms, and that these mechanisms have a genetic basis (see FIG. 1). The role for genetic factors in SIDS is highlighted by the fact that the incidence in families where one infant has died from SIDS is increased over five-fold, and the relative risk of SIDS among twins is approximately twice that of singletons adjusting for other perinatal factors. SUDC is over-represented in families with an elevated rate of febrile seizures, suggesting a genetically mediated seizure-related pathogenesis. Identifying genes playing a role in vulnerability to SUDP provides answers to families and promotes new avenues of research into the pathophysiology of SUDP and its prevention.

Although a major cause of child mortality, SUDP is largely confined to medical examiner and coroner systems where it has been insulated from new developments in biomedical discovery, limiting research on this diagnostic dilemma. A review of SUDP cases from 2012-2014 found that medical assessments in SUDP were highly variable, often incomplete, and typically lacking the expert input of pediatric pathologists. Genetic testing was rarely performed, and when performed, done only on the proband with limited phenotyping.

SUDP is designated a research priority for the Eunice Kennedy Shriver National Institute of Child Health and Development (NICHD), and the NICHD has prioritized the development of specific and sensitive predictive tests for identifying fetuses and infants at risk for SIDS. In the context of further evolution of the molecular autopsy, insights gained from genetic studies have the potential to advance the understanding of SUDP, potentially revealing underlying genetic mechanisms that will allow risk stratification for surviving family members and eventually identification of infants at risk in the general population with the goal of prevention of SUDP.

SUDP represents a constellation of undiagnosed diseases. Methods for identifying the underlying diseases involve extensive phenotyping and comprehensive genomic analysis.

DNA samples were obtained from pathological materials (spleen and brain) on the proband for whole exome sequencing (WES). DNA was obtained blood from the parents for trio analysis.

### Phenotyping Methods

Decedent history: A detailed phenotypic analysis of each SUDP case was carried out. A case history was obtained, with special attention to features associated with SUDP, including circumstances of death; coincident acute illness; general medical history including specific medical problems, growth and development; general physical findings; and family, obstetric, and birth histories. The neurological history, including febrile seizures, seizure or epilepsy history, head circumference, and neurological examination.

Family history: The family history includes, but is not limited to, sudden death, febrile seizures, epilepsy, neuro-developmental disorders, cardiac disease including arrhythmia, and other disorders. As autopsy findings and later genetic findings are uncovered, the family history is revisited as needed.

General autopsy review: A pediatric pathologist reviewed general autopsy findings and documented phenotypic correlations.

Neuropathological review: Detailed neuropathological analysis was carried out including assessment for developmental abnormalities of the cortex and hippocampus and review of neuropathological materials, including uncut whole brains, neuropathology reports, and microscopic sections of the brain and spinal cord. Hippocampal sections were scored according to 44 standardized microscopic features.

Synthesis of phenotype: The phenotypic features informed the genetic analyses.

### Example 2: Whole Exome Sequencing (WES) Analysis of SUDP Trios and Families

WES was performed on SUDP cases and available family members. Genomic DNA samples were subjected to paired-end sequencing (2×75 bp) on Illumina HiSeq 2000 or 4000 platforms to provide a mean coverage >100×, with >80% of the target bases having at least 20× coverage (Broad Institute, Cambridge, Mass.). All exome data were analyzed on parallel platforms at the Broad Institute, BCH, and Baylor College of Medicine (Codified Genomics) to enhance validity of the variant calls. Exome data from trios to identify rare (<0.01% minor allele frequency) protein-altering variants. FASTQ files were processed using GATK Best Practices recommendations optimized for Mendelian disease gene discovery. A Mendelian check confirmed parent-child relationships. Variants were classified regarding pathogenicity per ACMG guidelines, considering the type of change (truncation vs. missense), location in the gene, whether the variant is de novo vs. inherited, and categorization in the ClinVar database as available. Deleterious effects of missense variants were predicted using in-silico analyses (Meta-SVM, PolyPhen-2 and SIFT software packages), comparison to the literature, other databases, and large reference ‘control’ databases spanning over 153,000 ancestrally diverse exomes and genomes (ExAC, gnomAD, and BRAVO). Variants were visualized in the University of California Santa Cruz genome browser to interrogate conservation of amino acids as well as protein motifs surrounding amino acids. Variants considered pathogenic or likely pathogenic were confirmed by PCR. Phenome Central and Matchmaker Exchange were used to determine if other researchers identified similar variants in candidate genes.

Gene list: A “SUDP Gene Panel” was assembled, which included over 200 genes (Table 1) some of which appeared to be involved in sudden death, epilepsy, brain malformation, cardiac arrhythmias, and/or metabolic diseases. Other genes have never been previously implicated in sudden death (Table 2)

Initial analysis using the curated list: The SUDP Gene Panel was used to identify rare, protein-altering variants in these genes. In proband-only (without parental data) cases, the analysis was carried out using the list to identify candidate genes for SUDP that could be screened in the rest of the cohort. For example, variants annotated as pathogenic in ClinVar would be implicated despite lack of parental data. In cases with trio data, because of the possibility of decreased penetrance seen in epilepsy and cardiac arrhythmias, variants in genes in these categories inherited from a parent were considered.

Novel gene discovery: In trios without findings on the SUDP Gene Panel, de novo or compound heterozygous variants in novel genes were sought. Given the severity of the sudden death phenotype, the exome data was analyzed for potentially disease causing (rare, protein-altering) variants in each proband, hypothesizing a role for de novo variants or compound heterozygous variants.

Identification of de novo variants: In trios, the presence of rare, protein-altering de novo heterozygous variants was analyzed and the relevance of the genes and variants identified to SUDP was assessed, taking into account the history of each case. For example, for cases with a history of febrile seizures, variants in genes related to brain development or synaptic dysfunction were considered relevant.

Identification of compound heterozygous or recessive variants: In trios, the presence of two compelling potentially pathogenic variants in a given gene inherited in either a compound heterozygous fashion or homozygous fashion using a minor allele frequency threshold of 0.001 was evaluated. In-depth analysis of these variants for potential pathogenicity was as above but included assessment of whether these variants were present in control databases (e.g., ExAC) in the homozygous state, which essentially ruled them out as pathogenic in the probands. Inherited heterozygous variants were considered potentially pathogenic, even though the phenotype is sudden death and the parent from whom the variant is inherited is living, with incomplete penetrance if the variants are present in ClinVar or are in regions where other variants have been reported in association with disease (e.g., transmembrane domains of sodium channels present in the heart or brain and associated with familial conditions with incomplete penetrance).

### De Novo Burden Analysis

The burden of variants within phenotypic groups outlined above in related genes based on expression levels was evaluated. A pipeline for rare variant enrichment analysis was developed, using Plink and R software packages. A binomial test was used to assess for genes with a significant difference in frequency of damaging (LOF and deleterious missense) de novo variants in the cohort of cases vs. controls. Because the initial sample of 29 trios with WES lacked sufficient power to evaluate the global burden of de novo variation in SUDP, 61 proband-only cases were compared to 1100 healthy adult controls (Alzheimer's Disease Sequencing Project, ADSP), evaluating for rare (allele frequency <0.0001) loss of function variants (LOF) and deleterious missense (DM) variants. Based on the hypothesis that SUDP will involve genes related to brain development, epilepsy, or cardiac arrhythmias, genes with high heart expression (HHE), high brain expression (HBE), or both (HHE-HBE), were analyzed with high expression defined as the top quartile of level of expression in the fetal mouse. A difference was considered significant if the p-value was below the Bonferroni-corrected threshold (0.05/number of genes tested).

27 trios (proband and parents) were evaluated. This evaluation included performing whole exome sequencing (WES) on the trio. 3/6 cases with SIDS and ⅘ cases with SUDC showed neuropathological evidence of temporal lobe pathology; and a natural manner of death was determined in the vast majority. The total of 95 SUDP cases included 61 proband-only cases, 29 trios (child and both parents), 3 multiplex families with SUDC, and 2 families with SUDC and epilepsy.

### WES Analysis

Variants of interest (Table 3) were observed including some predicted to be pathogenic, likely pathogenic, or variants of uncertain significance (VUS).

| | | Disease | | | Predicted |
|---|---|---|---|---|---|
| Case ID | Gene | category | Protein Change | MAF | pathogenicity |
| 1 | SCN1A | E | Leu61Pro | 0 | LP |
| 2 | SCN1A | E | Gly682Val | 0 | LP |
| 3 | SCN1A | E | Leu1296Met | 0 | LP |
| | | | Glu1308Asp (in | 91/120552 | |
| | | | cis) | | |
| 4 | GABRG1 | E | Tyr351Ter | 0 | VUS |
| 5 | LGI1 | E | c.432- | 0 | VUS |
| | | | 4_432delCCins7 | | |
| 6 | SCN1B | E, LQTS | Trp179Ter | 0 | P |
| 7 | SCN3A | E | Ala1804Val | 1/122870 | VUS |
| 8 | CCL3 | E | Pro30Leu | 4/122954 | LP |
| 9 | SPTAN1 | E | Trp131Arg | 2/19302 | LP |
| 9 | MYLK2 | C | Asn278Ile | 0 | LP |
| 10 | CACNB4 | E | Lys205Arg | 0 | LP |
| 11 | KCNB2 | LQTS | Ser714Phe | 289/5000000 | VUS |
| 12 | JUP | ARVD | Ala571Thr | 0 | LP |
| 13 | RAF1 | C | Ser605Phe | 0 | VUS |
| 14 | AKAP9 | LQTS | Ile544_GLn545del | 2/122278 | VUS |
| 15 | KCNH2 | LQTS | Gly879Arg | 0 | LP |
| 16 | ANK2 | LQTS | Arg3906Trp | 138/122818 | P |
| 17 | TRPM4 | HB | Try103Cys | 40/122870 | LP |
| 18 | MIB1 | LVNC | Arg1582Gln | 1/99094 | VUS |
| 19 | SCN2B | AF | Val40Ile | 3/21519 | VUS |
| 20 | MYPN | C | Val1995Met | 36/19200 | P |
| 20 | DSP | ARVD | Arg1852His | 26/16618 | LP |
| 20 | MYH7B | C | Met538ValfsTer23 | 41/21457 | LP |
| 21 | MYH7 | C | Arg1606His | 2/21520 | LP |
| 22 | HCN4 | LQTS | Lys717Asn | 0 | LP |
| 23 | HADA | LCHAD | Gly642Val | 4/122962 | LP |
| | | | homozygous | | |

A variant in SCN1B (associated with arrhythmias and epilepsy) was identified in two siblings. A “likely pathogenic” variant in SCN1A (associated with epilepsy) was found in two siblings from a family in which one child died of SUDC after a history of atypical febrile seizures and a living sibling has severe generalized epilepsy resembling Dravet syndrome; the variant derived from the father, who was mosaic for the variant. Two additional cases with variants in SCN1A and hippocampal abnormalities, but no history of seizures were identified. The same variant in KCNB2 (associated with arrhythmias) was identified in four siblings of a family in which three children died of SIDS and a surviving sibling has a history of febrile seizures; and in an unrelated SUDC case.

### De Novo Burden Analysis

In 11 cases with hippocampal abnormalities, a significant enrichment of rare LOF variants was identified in HHE-HBE genes in cases vs. controls (OR 6.4, p=0.0016), and rare damaging variants (LOF or DM) in HBE genes in cases vs. controls (OR 1.3, p=0.0147). In 6 cases with a family history of cardiac disease, a significant enrichment of rare DM variants in HHE-HBE genes was found in cases vs. controls (OR 13.6, p=0.0108).

The findings of variants in SCN1A and SCN1B were consistent with a relationship between epilepsy and SUDP in many (about 40%) cases. SCN1A is implicated in Sudden Unexpected Death in Epilepsy (SUDEP) in patients with Dravet syndrome, and there may be a link between SUDP, SUDEP, and SCN1A.

The KCNB2 variant, found in two unrelated families, is associated with Brugada syndrome, a cardiac channelopathy characterized by ST-segment elevations in the anterior precordial leads of an ECG, and a high incidence of sudden death. Cardiac channelopathies, including Brugada syndrome, were also associated with SIDS, and may explain 10% of SIDS cases. The role of KCNB2, as a relatively new gene associated with Brugada syndrome, is unclear but the finding is very intriguing.

Insights gained from these genetic studies have the potential to advance the understanding of SUDP, revealing underlying genetic mechanisms that allow risk stratification for surviving family members and eventually identification of infants at risk in the general population with the goal of prevention of SUDP. Functional analysis of candidate variants will be a key next step for more deeply understanding the role of genetics in SUDP.

### Example 2: SCN1A Variants Identified in SIDS

Hippocampal abnormalities were identified in approximately 40% of infants dying of SIDS, chiefly bilamination of the granule cell layer in the dentate gyrus. This same lesion was unexpectedly identified in children over 1 year of age dying of Sudden Unexplained Death in Childhood (SUDC). Such lesions are classically associated with temporal lobe epilepsy.

The association of epilepsy-related pathology with SIDS and SUDC, recently called epilepsy in situ, leads to questions about epilepsy-related mechanisms in sudden death. Sudden Unexpected Death in Epilepsy (SUDEP) exemplifies the well-recognized association between epilepsy and sudden death. In addition, an association between SUDC and personal or family history of febrile seizures (FS) has been described, suggesting possible shared genetic predispositions for these entities. Notably, the SIDS infants and SUDC children with hippocampal abnormalities had not been diagnosed with epilepsy, though some had a history of FS. Collectively, these data support an association between sudden death and seizures, even in the absence of overt epilepsy.

Whole exome sequencing (WES) was performed to evaluate 10 cases of SIDS with the hypothesis that some cases are associated with epilepsy-associated genes. This resulted in the discovery of SCN1A variants in two SIDS cases using the following methods and materials.

DNA from 10 SIDS cases was obtained through the Office of the Medical Examiner (OME), San Diego, Calif. in accordance with California law Chapter 955, Statutes of 1989 (SB1069), permitting the use of autopsy tissues and DNA from SIDS infants for research. Samples were anonymous; parental samples are not available. Antemortem history and autopsy findings were reviewed for evidence of known causes of death.

WES and analysis were performed using standard methods plus evaluation for the presence of variants in three SCN1A-specific databases: SCN1A Variant Database, SCN1A Infobase, and the Ghangzhou Medical University SCN1A Database. Further analysis was carried out on variants with population allele frequency <0.001 and OMIM disease associations, particularly sudden death, seizures, cardiac arrhythmia, and metabolic disease. The American College of Medical Genetics and Genomics (ACMG) guidelines for variant interpretation was applied to each variant (Richards et al., Standards and Guidelines for the Interpretation of Sequence Variants: A Joint Consensus Recommendation of the American College of Medical Genetics and Genomics and the Association for Molecular Pathology; Genet Med 2015; 17:405-24; doi:10.1038/gim.2015.30). All variants considered likely pathogenic were confirmed by Sanger sequencing. Functional evaluation of the variants was performed using manual patch-clamp recording. Mutagenesis of recombinant human Na_{V}1.1 (encoded by SCN1A) was performed as previously described^{10,11} to create G692V and the compound variant L1296M/E1308D. The open reading frames of all plasmid preparations were sequenced in their entirety prior to use in experiments. Heterologous co-expression of WT Na_{V}1.1 or the SIDS-associated variants with the human (31 and (32 subunits in tsA201 cells was performed as previously described.¹¹ Whole-cell voltage clamp recording was performed at room temperature as previously described.^{11,12}

From among the 10 cases ascertained with SIDS, 5 had hippocampal sections available; of these, ⅗ had dentate gyrus abnormalities, including the two reported here.

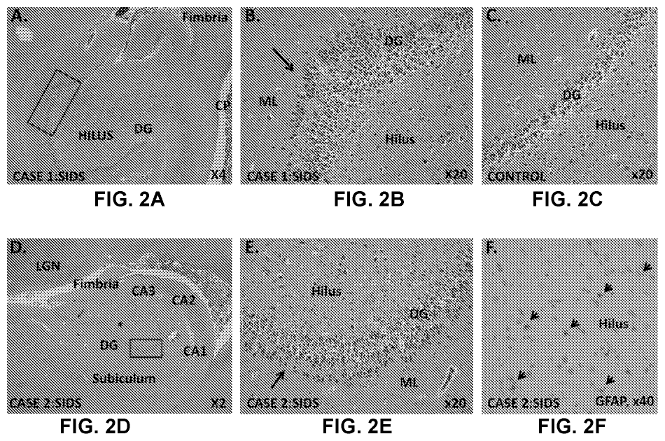

Case 1 was a Caucasian girl who died at 2 months, with cause of death recorded as SIDS. The infant had prenatal opioid exposure, a known risk factor for SIDS. She was born at 35 gestational weeks to an opioid-dependent mother who began methadone treatment at 5 gestational months. At birth, the infant required medication for neonatal opiate withdrawal syndrome for 19 days and subsequently metoclopramide and lansoprazole for gastroesophageal reflux disease (GERD). She was placed in foster care and had been healthy prior to death. Prior to death, she had been swaddled and placed supine to sleep, with the head of the bed elevated as recommended for GERD. She was found diaphoretic and unresponsive in the prone position. Toxicological assessment for drugs of abuse was negative. Neuropathological examination revealed no macroscopic abnormalities. Microscopic examination of the hippocampus revealed focal bilamination of the dentate gyrus (FIGS. 2A and 2B, compare FIG. 2C).

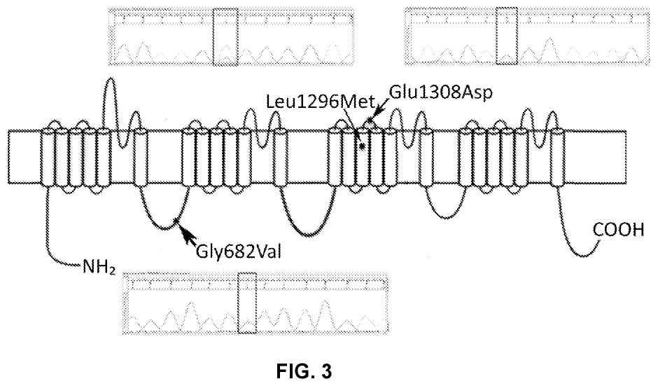

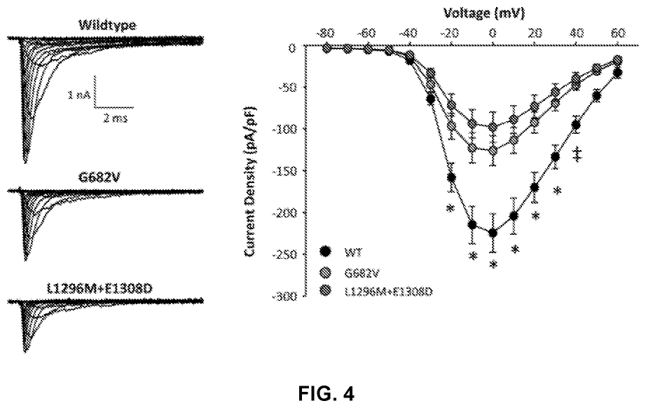

Exome analysis revealed SCN1A c.2045G>T, p.G682V (NM_001202435.1), confirmed by Sanger sequencing. Gly682 is a highly conserved amino acid in a cytoplasmic domain of SCN1A. SIFT score is 0 (deleterious), MutationTaster score is 1 (disease-causing), but Polyphen-2 score is 0.069 (benign). The variant is not seen in the ESP, ExAC, or the three referenced SCN1A databases, but reported nearby variants affecting the same domain have been associated with Dravet syndrome (D674G)¹³ and borderline severe myoclonic epilepsy of infancy, (T685LfsX5)¹⁴ (FIG. 3.) Functional evaluation of this variant demonstrated significantly lower current density compared with WT channels, consistent with a partial loss-of-function effect (FIG. 4); additional experiments showed no differences in the voltage-dependence of activation or inactivation, recovery from inactivation, or use-dependent channel rundown. No other variants were present that could plausibly explain the phenotype.

Case 2 was a Caucasian girl who died at age 7 weeks with cause of death reported as SIDS. The mother had received prenatal care beginning at 4.5 months gestation and was placed on bedrest at 6 months gestation due to potential placental abruption. The mother was positive for Group B Streptococcus (GBS); the infant's GBS status was not reported. Exposures were limited to second-hand tobacco smoke. The infant fell asleep in her caregiver's arms, was placed supine in an adult bed, and witnessed supine while sleeping. She was found prone and unresponsive. There were no macroscopic findings on neuropathological assessment. Examination of the hippocampi revealed focal areas of bilamination and a small amount of hilar gliosis (FIGS. 2D to 2F).

Exome analysis identified two SCN1A variants, c.3886T>A, p.L1296M and c. 3924A>T, p.E1308D, each confirmed by Sanger sequencing, and determined to be in cis configuration by direct inspection of the exome data in the Integrated Genomics Viewer (IGV). The L1296M variant affects the highly conserved L1296 amino acid in the SCN1A S3 helical loop of transmembrane domain III. SIFT score is 0 (deleterious), Mutation Taster score is 0.616 (polymorphism), and Polyphen-2 score is 0.897 (possibly damaging). The variant is not seen in the ESP, ExAC, or the referenced SCN1A databases but is in close proximity to a nonsense variant and an in-frame deletion associated with epilepsy (FIG. 3).

The c. 3924A>T, p.E1308D variant affects a highly conserved amino acid in the extracellular domain of the transmembrane domain III of SCN1A (FIG. 3). SIFT score is 0 (deleterious), MutationTaster score is 0.998 (disease-causing), and Polyphen-2 score is 0.042 (benign). The variant is present in ClinVar as a variant of uncertain significance (VUS), associated with Dravet syndrome. It is present in the ESP (ESP6500SIV2) in 0.09% of European Americans and in ExAC with allele frequency 0.075%. This variant has also been reported in association with familial febrile seizures and Dravet syndrome, including in a child with a variant inherited from an asymptomatic parent, (referenced in SCN1A Infobase and Ghangzhou SCN1A database). Functional assessment of this compound variant (L1296M/E1308D) demonstrated lower whole-cell sodium current density compared to WT channels to a degree similar to G682V, consistent with a partial loss-of-function (FIG. 4); additional experiments demonstrated no differences in the voltage-dependence of activation or inactivation, recovery from inactivation, or use-dependent channel rundown.

Exome data analysis also revealed a variant in AKAP9, NM_005751, ENST00000356239.3:c.1924G>A, p.Glu642Lys, predicted pathogenic. However, the gene is tolerant to missense variation (ExAC missense constraint metric z=−2.75), and the variant is not in proximity to the KCNQ1-binding domains of AKAP9 or the single published long QT syndrome-associated variant. Therefore, we conclude that the AKAP9 variant is not a contributor to SIDS in this case. No other disease-associated variants related to sudden death were present for this case.

The present analysis revealed a novel association between SCN1A and SIDS, evidence for a role for genetics in SIDS. From a cohort of 10 infants with SIDS, two cases with heterozygous SCN1A variants were identified. The variants are predicted to be pathogenic using in silico assessments. These predictions are further strengthened by association with previously reported cases with epilepsy, location of the variants in critical, disease-associated domains of the protein, and functional evidence that the variants present in both cases exhibit partial loss-of-function. SCN1A encodes Na_{V}1.1, a voltage-gated sodium channel, expressed in human brain during fetal and early post-natal life. SCN1A variants are associated with the familial syndrome Genetic Epilepsy with Febrile Seizures Plus (GEFS+), with a wide phenotypic spectrum from unaffected or mildly affected with febrile seizures to severe epileptic encephalopathy. SCN1A is also associated with Dravet syndrome (severe myoclonic epilepsy of infancy), typically with de novo heterozygous truncating or missense mutations, with both types affecting the same translated protein domains. SCN1A is intolerant to missense variation (ExAc constraint metric z-score=5.61), and its role in a clinically diverse group of epilepsies was highlighted in the largest genome-wide association study of epilepsy.²¹ Although we are unable to determine whether the variants in the two cases reported here are de novo or inherited because of lack of access to parental DNA, given the wide range of phenotypes associated with this gene and the demonstrated loss of function associated with these variants, the lack of parental data does not diminish the impact of our findings.

A unique feature of the two cases with SIDS and SCN1A variants is hippocampal dentate gyrus bilamination, a variant of granule cell dispersion classically associated with temporal lobe epilepsy. This feature has been described previously in association with SIDS and SUDG but not with SCN1A prior to this report. The limited literature on neuropathological abnormalities in patients with SCN1A-related epilepsy includes hippocampal sclerosis, focal cortical dysplasia, periventricular heterotopia, micronodular dysplasia of the medial temporal lobe, and granule cell dispersion of the dentate gyrus. Dentate bilamination, as seen in our two cases without overt epilepsy before death, may represent a primary developmental lesion and may represent an epileptogenic nidus for the generation of seizures, in these cases subclinical. Alternatively, the dentate bilamination may be secondary to seizures, again subclinical, that arose in the hippocampus due to SCN1A dysfunction. The extent to which SCN1A and other epilepsy-related genes are associated with the developmental hippocampal abnormalities observed in 40-50% of cases with SIDS and SUDC, remains to be determined.

SCN1A variants in the cases reported represent an intrinsic vulnerability that, in combination with other endogenous and exogenous factors, contributed to the risk of SIDS. The association between SCN1A and SIDS extends the spectrum of SCN1A from febrile seizures and epilepsy to sudden death. Notably, infants and children classified as SIDS and SUDC with the SCN1A variants and dentate gyral lesions did not have a reported history of epilepsy. The novel association of SCN1A with SIDS supports further intense efforts to understand epilepsy-related mechanisms into sudden death across the age spectrum in individuals with and without an overt history of seizures or epilepsy.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A panel comprising one or more Sudden Unexpected Death in Pediatrics (SUDP) polynucleotides of Table 2 or fragments thereof fixed to a substrate.

2. The panel of claim 1, further comprising a SUDP polynucleotide of Table 1 or a fragment thereof, wherein each of polynucleotides of Table 1, Table 2, or fragments thereof are fixed to a substrate.

3. A set of primers or probes each of which selectively hybridizes to a SUDP polynucleotide or fragment thereof of claim 2.

4. A polynucleotide probe that hybridizes to a SUDP polynucleotide of claim 2.

5. A primer pair that hybridizes to and amplifies a SUDP polynucleotide of claim 2.

6. A polynucleotide array for characterizing SUDP, said array comprising at least ten probes immobilized on a substrate, each of said probes being between about 15 and about 500 nucleotides in length, each of said probes being derived from a sequence corresponding to, or complementary to, a transcript of a SUDP polynucleotide of Table 1 or Table 2.

7. A method of characterizing a plurality of SUDP polynucleotides in a pediatric subject who died suddenly and unexpectedly, the method comprising
(a) sequencing a plurality of SUDP polynucleotides of Table 1 and or Table 2, or fragments thereof, in a biological sample derived from the subject, and
(b) detecting the presence or absence of an alteration in the SUDP sequence relative to a reference sequence.

8. The method of claim 7, further comprising evaluating exome data to identify rare protein-altering variants.

9. The method of claim 7, further comprising evaluating the allele frequency.

10. The method of claim 7, wherein detection of an alteration in a SUDP polynucleotide identifies a cause of death for the subject.

11. (canceled)

12. The method of claim 7, further comprising analyzing one or more factors selected from the group consisting of circumstances of the death of the subject, coincident acute illness, specific medical problems, growth history, developmental history, general physical findings, family history, obstetric and birth history.

13. The method of claim 7, further comprising analyzing the subject's neurological history.

14. The method of claim 13, wherein neurological history includes febrile seizures, seizure or epilepsy history, head circumference, and neurological examination.

15. The method of claim 7, further comprising carrying out neuropathological, metabolic, and cardiac function testing, cytology, histology, ultrasound analysis, MRI results, CT scan results, and measurements of other biomarker levels.

16. The method of claim 15, wherein the neuropathological analysis is of the hippocampus, medulla, or amygdala.

17. A method of characterizing a plurality of SUDP polynucleotides in two or more related subjects, the method comprising
(a) sequencing a plurality of SUDP polynucleotides of claim 2, or fragments thereof, in a biological sample derived from the subjects,
(b) detecting the presence or absence of alterations in the SUDP sequences relative to a reference sequence; and
(c) analyzing inheritance among the subjects.

18. The method of claim 17, wherein the related subjects are a proband and their siblings.

19. The method of claim 17, wherein the related subjects are a proband and parents.

20. A kit for characterizing a plurality of SUDP polynucleotides of claim 2, the kit comprising one or more of primers, probes, panels, or arrays.

21. A method of treating a subject at risk of SUDP, the method comprising identifying an alteration in a gene associated with QT syndrome in the subject and administering a beta blocker to said subject.
